(19) United States
(12) Patent Application Publication
(10) Pub. No.: US 2007/0088163 A1
(43) Pub. Date: Apr. 19, 2007

(54) Modulators of calcitonin and amylin activity

(75) Inventors: Kent Pryor, San Diego, CA (US); Eddine Saiah, Brookline, MA (US); Jeffrey D. Kahl, San Antonio, TX (US); Nancy G. J. Delaet, San Diego, CA (US); Edward Roberts, Fallbrook, CA (US); Jan Urban, San Diego, CA (US); Lubomir Sebo, San Diego, CA (US); Christopher Lum, San Diego, CA (US); Hiroshi Nakanishi, San Diego, CA (US)

Correspondence Address: FOLEY & LARDNER LLP, P.O. BOX 80278, SAN DIEGO, CA, 92138-0278, US

(73) Assignee: Kemia, Inc.

(21) Appl. No.: 10/571,031

(22) PCT Filed: Sep. 10, 2004
(86) PCT No.: PCT/US04/29385

§ 371 Date: Dec. 26, 2006

Related U.S. Application Data
(60) Provisional application No. 60/502,402, filed on Sep. 12, 2003.

(51) Int. Cl. C07D 471/02; C07D 215/16

(52) U.S. Cl. 546/113; 546/177

(57) ABSTRACT

In various aspects, the present invention relates to novel compounds, which modulate calcitonin and amylin receptor activity; to processes for the preparation of such compounds; and to pharmaceutical compositions including such compounds. Compounds of the invention are useful as calcitonin and/or amylin agonists and in the treatment of bone disease such as osteoporosis, Paget's disease, hypercalcemia, and in the treatment of metabolic diseases, such as insulin-requiring states.

### FIELD OF THE INVENTION

The present invention relates to low molecular weight compounds and pharmaceutical compositions thereof, useful as modulators of calcitonin and amylin activity. In particular, the compounds are well suited for use as calcitonin and/or amylin agonists. The invention further relates to methods of preparation and use of such compounds and compositions in treating disorders mediated by calcitonin and amylin receptors such as bone and metabolic diseases.

### BACKGROUND OF THE INVENTION

Bone is a dynamic tissue, and homeostasis in the adult skeleton requires a balance between bone resorption and bone formation. Osteoclasts and osteoblasts play a key role in this balance, with osteoclasts initiating bone resorption and osteoblasts synthesizing and depositing new bone matrix. Imbalances in bone homeostasis are associated with such disorders as osteoporosis, Paget's disease, and hyperparathyroidism.

The activities of osteoclasts and osteoblasts are regulated by complex interactions between systemic hormones and the local production of growth factors and cytokines. Calcitonin, a peptide hormone secreted by the thyroid and thymus of mammals, plays an important role in maintaining bone homeostasis. Calcitonin inhibits bone resorption through binding and activation of a specific calcitonin receptor on osteoclasts [M. Pondel, Calcitonin and Calcitonin Receptors: Bone and Beyond, Int. J. Exp. Path. 81, 405-22 (2000)], with a resultant decrease in the amount of calcium released by bone into the serum. This inhibition of bone resorption has been exploited, for instance, by using calcitonin as a treatment for osteoporosis, a disease characterized by a decrease in the skeletal mass often resulting in debilitating and painful fractures. Calcitonin is also used in the treatment of Paget's disease where it provides rapid relief from bone pain, which is frequently the primary symptom associated with this disease. This analgesic effect has also been demonstrated in patients with osteoporosis or metastatic bone disease and has been reported to relieve pain associated with diabetic neuropathy, cancer, migraine and post-hysterectomy. Reduction in bone pain occurs before the reduction of bone resorption.

Despite the frequent use of animal-derived calcitonin in humans (such as salmon calcitonin), there are many disadvantages. For treatment of osteoporosis, for instance, daily prophylactic administration can be required for 5 or more years at a relatively high cost. In the United States, calcitonin is often administered by injection, and since the disease indications for this drug are not usually life threatening, patient compliance can be low. Resistance to calcitonin therapy may occur with long-term use. In addition, some patients develop antibodies to non-human calcitonin.

Amylin and adrenomedullin are related peptides with some homology to both calcitonin and calcitonin gene-related peptide. [P M. Sexton et al., Curr. Med. Chemistry, 1999, 6:1067] Like the peptides themselves, the receptors for the calcitonin family are related to each other. Each peptide acts through its own distinct high affinity receptor, as well as through other receptors of the family, usually with lower affinity. Amylin and adrenomedullin have recently been found to stimulate the proliferation of osteoblasts in vitro, and to increase indices of bone formation when administered either locally or systemically in vivo. Furthermore, amylin inhibits bone resorption. [Cornish, J. et al., Curr. Pharm. Des., 2002, 8(23):2009]. In addition to its bone-effects, however, amylin also has an important role in postprandial glucose regulation. The neuroendocrine hormone is cosecreted with insulin at mealtimes and has been shown to suppress postprandial glucagon secretion [Gedulin, B. et al., Metabolism, 1997, 46:67], to regulate gastric emptying [Young, A. et al., Diabetologia, 1995, 38:642] and to reduce food intake [Rushing et al., Endocrinology, 2000, 141:850]. Because of these effects, the peptide and related analogues such as pramlintide are being investigated for the treatment of diabetes. Similar to calcitonin, the drawback to this peptidic approach is the need for administration by injection, resulting in potentially reduced patient compliance and high costs of production. Accordingly, there is a need in the art for small molecule compounds that can modulate the activity of calcitonin and/or amylin receptors and which are useful in the treatment of bone and metabolic diseases mediated by calcitonin and amylin receptors. Moreover there is a need for such compounds which overcome one or more of the disadvantages described above.

### SUMMARY OF THE INVENTION

In various aspects, the present invention relates in part to compounds, having Formula I; to processes for preparing compounds of Formula I; to pharmaceutical compositions including such compounds; and to methods of use and treatment with such compounds. More specifically, bicyclic heterocycles including a pyridinyl ring are provided for use in the treatment disorders mediated by calcitonin and/or amylin.

Thus, there are provided compounds having formula I, 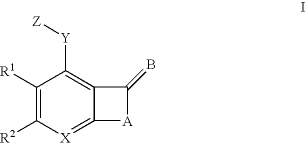
stereoisomers thereof, tautomers thereof, solvates thereof, and pharmaceutically acceptable salts thereof; wherein

A is —NH—CR^{3a}R^{3b}— wherein the NH is attached to the carbon bearing B, or A is —CRR′—CR^{4a}R^{4b}—CRR′—;

B is O or S;

X is N or NO;

Y is a divalent substituted or unsubstituted aryl, heterocyclyl, or cycloalkyl group;
Z is —C(O)OR⁵, —C(O)NR⁶R⁷, —NR⁶C(O)R⁵, —NR⁶C(O)NR⁶R⁷, —C(O)R⁵, —NR⁶R⁷, —OR⁸, —SO₂NR⁶R⁷, —NR⁶SO₂R⁵ or —S(O)ₘR⁵;

R and R′ at each occurrence are independently H or an unsubstituted C₁₋₃ alkyl group;

R¹ is H, —C(O)OR⁹, —C(O)NR¹⁰R¹¹, —CN, —C(O)R⁹ or —NR¹⁰C(O)R⁹;

R² is —(C₁₋₂ alkyl)-R¹² wherein the C₁₋₂ alkyl is substituted unsubstituted;

R^{3a} is a C₂₋₃ alkyl group, optionally substituted with one or more F;

R^{3b} is H or a C₂₋₃ alkyl group, optionally substituted with one or more F;

R^{4a} and R^{4b} are each independently —H, or substituted or unsubstituted C₁₋₆ alkyl;

R⁵ a substituted or unsubstituted aralkyl, heteroaralkyl, heterocyclylalkyl, alkyl-cycloalkyl, fused cycloalkylaryl, or fused heterocyclylaryl group;

R⁶ and R⁷ are each independently —H or a substituted or unsubstituted aralkyl, heteroaralkyl, heterocyclylalkyl, alkyl-cycloalkyl, fused cycloalkylaryl, or fused heterocyclylaryl group; or R⁶ and R⁷, when attached to the same atom, together form a substituted or unsubstituted heterocyclyl group;

R⁸ is a substituted or unsubstituted, branched or unbranched C₂₋₆ alkyl, C₂₋₆ alkenyl, C₂₋₆ alkynyl or C₇₋₁₀ aralkyl group;

R⁹ is —H or a substituted or unsubstituted alkyl, heteroalkyl, alkenyl, alkynyl, aryl, aralkyl, heterocyclyl, or heterocyclylalkyl group;

R¹⁰ and R¹¹ are each independently —H or a substituted or unsubstituted alkyl, heteroalkyl, alkenyl, alkynyl, aryl, aralkyl, heterocyclyl, heterocyclylalkyl group; or R¹⁰ and R¹¹, when attached to the same atom, together form a substituted or unsubstituted heterocyclyl group;

R¹² is a substituted or unsubstituted cycloalkyl, aryl or heteroaryl group or an unsubstituted alkyl group; and

m is 0, 1 or 2.

As indicated by Formula I, compounds of the invention include bicyclic heterocycles having as one of the rings a pyridinyl (X is N) group or the N-oxide thereof. In some embodiments, A is —NH—CR^{3a}R^{3b}—, in which NH is attached to the carbon bearing B, and the present compounds are dihydropyrrolopyridines. In others, A is —CRR′—CR^{4a}R^{4b}—CRR′—, and the compounds are dihydroquinolines. In some embodiments, B is O, providing dihydropyrrolopyridinones and dihydroquinolinones.

In some embodiments of compounds of Formula I, Y is a divalent substituted or unsubstituted aryl or heterocyclyl group. By divalent it is meant that Y is attached to both Z and the bicyclic ring as indicated in Formula I. Typically, Y is a divalent aryl or heteroaryl, e.g., a divalent phenyl or pyridinyl group.

In other embodiments of compounds of Formula I, Z is —C(O)OR⁵, —C(O)NR⁶R⁷, —NR⁶C(O)R⁵, —C(O)R⁵, —OR⁸, —SO₂NR⁶R⁷, —NR₆R₇SO₂R₅ or —S(O)ₘR⁵. Alternatively, Z can be —C(O)OR⁵, —C(O)NR⁶R⁷, or —OR⁸.

As shown above, compounds of Formula I are substituted with R¹ and R². In some embodiments R¹ is H, —C(O)OR⁹ or —C(O)NR¹⁰R¹¹. In other embodiments, R⁹, R¹⁰ and R¹¹ are each independently —H, methyl, ethyl, n-propyl, n-butyl, isobutyl, sec-butyl, tert-butyl, cyclopentyl, or allyl. In still other embodiments, R² is —CH₂—CH₂—R¹². Exemplary groups contemplated for use as R¹² include but are not limited to cyclohexyl, cyclopentyl, phenyl, 4-fluorophenyl, 4-trifluorophenyl, isobutyl or t-butyl.

In certain embodiments, compounds of Formula I have the Formula II:

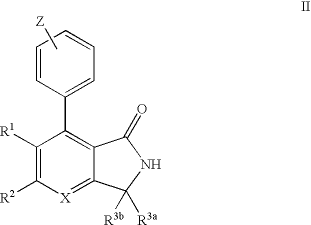

In such compounds, the variables X, Z, R¹, R², R^{3a} and R^{3b} are as defined for compounds of Formula I. In some such embodiments, Z is —C(O)OR⁵, —C(O)NR⁶R⁷, or —OR⁸.

In certain embodiments, compounds of Formula I have the Formula III:

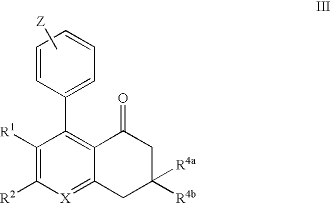

In such compounds, the variables X, Z, R¹, R^{3a} and R^{3b} are as defined for compounds of Formula I. In some such embodiments, Z is —C(O)OR⁵, —C(O)NR⁶R⁷, or —OR⁸.

Representative compounds of the invention include
2-[2-(4-Fluoro-phenyl)-ethyl]-4-{4-[(furan-2-ylmethyl)-carbamoyl]-phenyl}-7-isopropyl-5-oxo-6,7-dihydro-5H-pyrrolo[3,4-b]pyridine-3-carboxylic acid ethyl ester,
4-{2-[2-(4-Fluoro-phenyl)-ethyl]-7-isopropyl-5-oxo-6,7-dihydro-5H-pyrrolo[3,4-b]pyridin-4-yl}-N-furan-2-ylmethyl-benzamide,
2-[2-(4-Fluoro-phenyl)-ethyl]-4-{4-[(furan-2-ylmethyl)-carbamoyl]-phenyl}-7,7-dimethyl-5-oxo-5,6,7,8-tetrahydro-quinoline-3-carboxylic acid ethyl ester,
2-(2-Cyclohexyl-ethyl)-4-{4-[(furan-2-ylmethyl)-carbamoyl]-phenyl}-7-isopropyl-5-oxo-6,7-dihydro-5H-pyrrolo[3,4-b]pyridine-3-carboxylic acid ethyl ester,
2-(2-Cyclohexyl-ethyl)-4-{4-[(furan-2-ylmethyl)-carbamoyl]-phenyl}-7-isopropyl-5-oxo-6,7-dihydro-5H-pyrrolo[3,4-b]pyridine-3-carboxylic acid methyl ester,
2-(2-Cyclohexyl-ethyl)-4-{4-[(furan-2-ylmethyl)-carbamoyl]-phenyl}-7-isopropyl-5-oxo-6,7-dihydro-5H-pyrrolo[3,4-b]pyridine-3-carboxylic acid allyl ester,
2-(2-Cyclohexyl-ethyl)-4-{4-[(furan-2-ylmethyl)-carbamoyl]-phenyl}-7-isopropyl-5-oxo-6,7-dihydro-5H-pyrrolo[3,4-b]pyridine-3-carboxylic acid isopropyl ester,
2-(2-Cyclohexyl-ethyl)-4-{4-[(furan-2-ylmethyl)-carbamoyl]-phenyl}-7-isopropyl-5-oxo-6,7-dihydro-5H-pyrrolo[3,4-b]pyridine-3-carboxylic acid butyl ester,
2-(2-Cyclohexyl-ethyl)-4-{4-[(furan-2-ylmethyl)-carbamoyl]-phenyl}-7-isopropyl-5-oxo-6,7-dihydro-5H-pyrrolo[3,4-b]pyridine-3-carboxylic acid cyclopentyl ester,
2-(2-Cyclohexyl-ethyl)-4-{4-[(furan-2-ylmethyl)-carbamoyl]-phenyl}-7-isopropyl-5-oxo-6,7-dihydro-5H-pyrrolo[3,4-b]pyridine-3-carboxylic acid propyl ester,
2-(2-Cyclohexyl-ethyl)-4-{4-[(furan-2-ylmethyl)-carbamoyl]-phenyl}-7-isopropyl-5-oxo-6,7-dihydro-5H-pyrrolo[3,4-b]pyridine-3-carboxylic acid isobutyl ester,
4-{4-[(Furan-2-ylmethyl)-carbamoyl]-phenyl}-7-isopropyl-5-oxo-2-[2-(4-trifluoromethyl-phenyl)-ethyl]-6,7-dihydro-5H-pyrrolo[3,4-b]pyridine-3-carboxylic acid ethyl ester,
2-(4-Fluoro-benzyl)-4-{4-[(furan-2-ylmethyl)-carbamoyl]-phenyl}-7-isopropyl-5-oxo-6,7-dihydro-5H-pyrrolo[3,4-b]pyridine-3-carboxylic acid ethyl ester,
4-{4-[(Furan-2-ylmethyl)-carbamoyl]-phenyl}-7-isopropyl-5-oxo-2-phenethyl-6,7-dihydro-5H-pyrrolo[3,4-b]pyridine-3-carboxylic acid ethyl ester,
2-[2-(4-Fluoro-phenyl)-ethyl]-4-[4-(indan-1-ylcarbamoyl)-phenyl]-7-isopropyl-5-oxo-6,7-dihydro-5H-pyrrolo[3,4-b]pyridine-3-carboxylic acid ethyl ester,
2-[2-(4-Fluoro-phenyl)-ethyl]-4-{4-[(furan-2-ylmethyl)-carbamoyl]-phenyl}-7-methyl-5-oxo-5,6,7,8-tetrahydro-quinoline-3-carboxylic acid ethyl ester,
(S)-7-Ethyl-2-[2-(4-fluoro-phenyl)-ethyl]-4-{4-[(furan-2-ylmethyl)-carbamoyl]-phenyl}-5-oxo-6,7-dihydro-5H-pyrrolo[3,4-b]pyridine-3-carboxylic acid ethyl ester,
2-(2-Cyclopentyl-ethyl)-4-{4-[(furan-2-ylmethyl)-carbamoyl]-phenyl}-7-isopropyl-5-oxo-6,7-dihydro-5H-pyrrolo[3,4-b]pyridine-3-carboxylic acid ethyl ester,
2-[2-(4-Fluoro-phenyl)-ethyl]-7-isopropyl-5-oxo-4-{4-[(pyridin-3-ylmethyl)-carbamoyl]-phenyl}-6,7-dihydro-5H-pyrrolo[3,4-b]pyridine-3-carboxylic acid ethyl ester,
4-(4-tert-Butoxy-phenyl)-2-[2-(4-fluoro-phenyl)-ethyl]-7-isopropyl-5-oxo-6,7-dihydro-5H-pyrrolo[3,4-b]pyridine-3-carboxylic acid ethyl ester
4-[4-(4-Fluoro-benzyloxy)-phenyl]-2-[2-(4-fluoro-phenyl)-ethyl]-7-isopropyl-5-oxo-6,7-dihydro-5H-pyrrolo[3,4-b]pyridine-3-carboxylic acid ethyl ester,
4-[2-(2-Cyclohexyl-ethyl)-7-isopropyl-5-oxo-6,7-dihydro-5H-pyrrolo[3,4-b]pyridin-4-yl]-N-furan-2-ylmethyl-benzamide,
(S)-2-[2-(4-Fluoro-phenyl)-ethyl]-4-{6-[(furan-2-ylmethyl)-carbamoyl]-pyridin-3-yl}-7-isopropyl-5-oxo-6,7-dihydro-5H-pyrrolo[3,4-b]pyridine-3-carboxylic acid ethyl ester,
2-(2-Cyclohexyl-ethyl)-4-[4-(indan-1-ylcarbamoyl)-phenyl]-7-isopropyl-5-oxo-6,7-dihydro-5H-pyrrolo[3,4-b]pyridine-3-carboxylic acid ethyl ester,
2-(2-Cyclohexyl-ethyl)-4-[4-(3,4-difluoro-benzylcarbamoyl)-phenyl]-7-isopropyl-5-oxo-6,7-dihydro-5H-pyrrolo[3,4-b]pyridine-3-carboxylic acid ethyl ester,
N-Furan-2-ylmethyl-4-(7-isopropyl-5-oxo-2-phenethyl-6,7-dihydro-5H-pyrrolo[3,4-b]pyridin-4-yl)-benzamide, and
4-[(S)-2-(2-Cyclohexyl-ethyl)-7-isopropyl-5-oxo-6,7-dihydro-5H-pyrrolo[3,4-b]pyridin-4-yl]-N-indan-1-yl-benzamide.

In another aspect, the invention provides methods of preparing compounds of Formula I. For example, when A is —NH—CR^{3a}R^{3b}— and X is N in Formula I, the methods include

exposing a compound of formula IV to a first oxidant that selectively oxidizes a dihydropyridinyl ring to a pyridinyl ring,

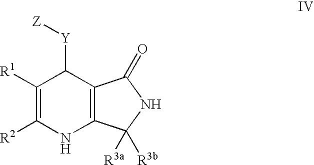

thereby providing a compound of formula IA

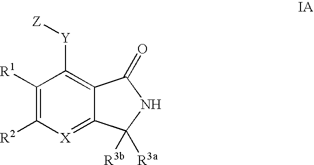

wherein Y, Z, R¹, R², R^{3a}, and R^{3b} are as defined previously.

Suitable first oxidants for use in the transformation of IV to IA include ceric (IV) ammonium nitrate (CAN), 2,3-dichloro-5,6-dicyano-1,4-benzoquinone (DDQ), Pd/acetic acid, [bis(trifluoroacetoxy)iodo]benzene and the like. In some embodiments of the method, Y is a divalent phenyl or pyridinyl group. In others, Z is —C(O)OR⁵, —C(O)NR⁶R⁷, or —OR⁸.

In some embodiments of methods of making compounds of Formula I, the methods include the step of preparing the compound of Formula IV by reacting Z-Y—CHO, R¹CH═C(NH₂)R², and a tetramic acid having the following structure

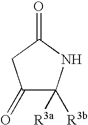

in the presence of ammonia to provide the compound of formula IV.

In these methods, any suitable source of ammonia may be used such as but not limited to ammonium hydroxide, ammonium acetate, ammonium carbonate.

The methods of making compounds of Formula I may further include the step of exposing the compound of Formula IA to a second oxidant that selectively oxidizes the pyridinyl N to an N-oxide, thereby providing a compound of Formula IB:

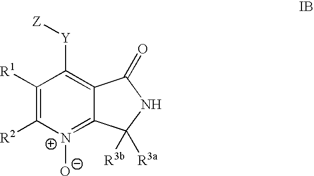

Second oxidants that may be used include but are not limited to hydrogen peroxide and m-chloroperbenzoic acid (MCPBA).

Other methods of preparing compounds of Formula I wherein A is —NH—CR^{3a}R^{3b}— and X is N include

exposing a compound having formula V to conditions suitable for removing PG²,

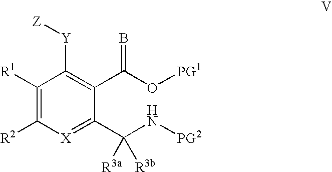

thereby producing a compound having the formula IA,

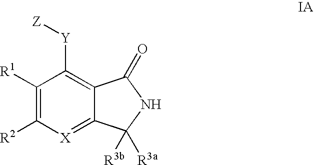

wherein Y, Z, R¹, R², R^{3a}, and R^{3b} are as defined previously and PG¹ and PG² are protecting groups.

There are further provided methods of preparing compounds of Formula I wherein A is —CRR′—CR^{4a}R^{4b}—CRR′—, X is N, and Y is aryl or heteroaryl, the methods including

exposing a compound of formula VI to an oxidant

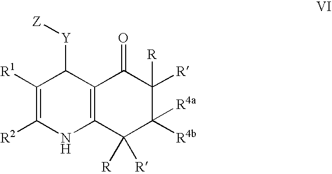

to provide a compound of formula IC,

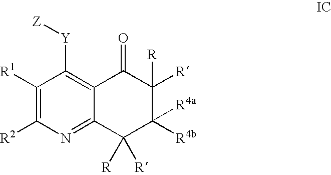

In some such embodiments of the methods, Y is a divalent phenyl or pyridinyl group. In others Z is —C(O)OR⁵, —C(O)NR⁶R⁷, or —OR⁸. Any suitable first oxidant as described herein may be used.

In some embodiments, the methods further include the step of preparing the compound of Formula VI by reacting Z-Y—CHO, R¹CH═C(NH₂)R², and a cyclohexene-1,3-dione having the following structure

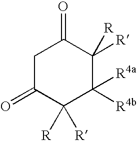

in the presence of ammonia to provide a compound of formula VI.

Methods of making compounds of Formula IC may further include the step of exposing the compound of Formula IC to a second oxidant that selectively oxidizes the pyridinyl N to N-oxide, thereby providing a compound of Formula ID:

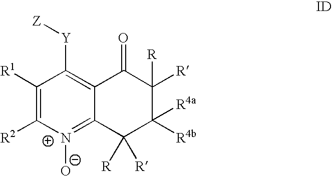

Suitable second oxidants are the same as described herein.

In accordance with yet another aspect of the invention, there are provided pharmaceutical compositions comprising a compound as described herein (e.g., a compound of Formulas I, II, or III) and a pharmaceutically acceptable carrier.

In a further aspect, the invention provides methods of preventing or treating a disorder mediated by calcitonin and/or amylin receptors, the method comprising administering to a subject in need thereof a therapeutically effective amount of a compound as described herein. In some such embodiments the compound administered is a calcitonin and/or amylin receptor agonist. Thus, compounds of the invention may be used to treat, bone disorders, metabolic disorders, pain, and other disorders, Exemplary bone disorders include osteoporosis, Paget's disease, hypercalcemia, Sudeck's atrophy, polystatic fibrous displasia, intersemocostoclavicular ossification, osteogenesis imperfecta, osteopenia, periodontal disease or defect, osteolytic bone disease, metastatic bone disorder, bone loss resulting from malignancy, autoimmune arthritides, breakage and fracture, or immobility and disuse. In some such embodiments, the bone disorder is osteoporosis or Paget's disease. Types of pain that may be treated using inventive compounds include, osteopathic pain, phantom limb pain, general pain, hyperalgesia, and pain associated with diabetic neuropathy. Exemplary metabolic disorders include insulin dependent diabetes, non-insulin dependent diabetes, impaired glucose tolerance, obesity, syndrome X, and diabetic complications. Other disorders amenable to treatment with inventive compounds include primary or secondary hyperthyroidism, endocrine disorder, conditions associated with inhibiting gastric secretion, gastrointestinal disorders, renal osteodystrophy, obesity, and male infertility.

There are further provided methods of modulating the activity of a calcitonin and/or amylin receptor including contacting the calcitonin and/or amylin receptor with a compound as described herein. The compound is typically an agonist at the calcitonin and/or amylin receptor.

Compounds of the invention can be used in combination with other therapies. In particular, compounds of this invention can be combined with insulin for the treatment of diabetes mellitus and other insulin-requiring states. By “insulin” is meant a polypeptide or its equivalent useful in regulation of blood glucose levels. A general description of such insulins is provided in Goodman and Gilman's The Pharmacological Basis of Therapeutics, 8th Ed., Pergamon Press (1990). Such insulins can be fast acting, intermediate acting, or long acting. Various derivatives of insulin exist and are useful in this invention. See, e.g., U.S. Pat. Nos. 5,049,547, 5,028,587, and 5,016,643. Insulin peptides are also useful (see, e.g., U.S. Pat. No. 5,008,241), as are analogues (see, e.g., U.S. Pat. Nos. 4,992,417 and 4,992,418). Such compositions can be administered by any standard route, including nasal administration (see, e.g., U.S. Pat. Nos. 4,988,512 and 4,985,242, and 2 BioWorld Today, No. 125 (1991)). The compounds of this invention are also useful in combination with a glucagon for the prevention and treatment of hypoglycemia. See Young et al., U.S. application Ser. No. 07/640,478, filed Jan. 10, 1991, entitled “Hyperglycemic Compositions.”

### DETAILED DESCRIPTION OF THE INVENTION

The following terms are used throughout as defined below.

Generally, reference to a certain element such as hydrogen or H is meant to include all isotopes of that element. For example, if an R group is defined to include hydrogen or H, it also includes deuterium and tritium.

The phrase “unsubstituted alkyl” refers to alkyl groups that do not contain heteroatoms. Thus the phrase includes straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, —CH(CH₂CH₃)CH(CH₃)CH(CH₃)(CH₂CH₃), and others. The phrase also includes cyclic alkyl groups such as cyclopropyl, cyclobutyl, cyclopentyl, cyclohexyl, cycloheptyl, and cyclooctyl and such rings substituted with straight and branched chain alkyl groups as defined above. Thus unsubstituted alkyl groups include cyclohexylethyl and cyclopentylmethyl groups, among others. The phrase also includes polycyclic alkyl groups such as, but not limited to, adamantyl, norbornyl, and bicyclo[2.2.2]octyl and such rings substituted with straight and branched chain alkyl groups as defined above. Thus, the phrase unsubstituted alkyl groups includes primary alkyl groups, secondary alkyl groups, and tertiary alkyl groups. Unsubstituted alkyl groups may be bonded to one or more carbon atom(s), oxygen atom(s), nitrogen atom(s), and/or sulfur atom(s) in the parent compound. Typical unsubstituted alkyl groups include straight and branched chain alkyl groups and cyclic alkyl groups having 1 to 20 carbon atoms, and more typical groups have from 1 to 10 carbon atoms. Even more typical groups, also known as unsubstituted lower alkyl groups, have from 1 to 5 carbon atoms. Most typical unsubstituted alkyl groups include straight and branched chain alkyl groups having from 1 to 3 carbon atoms and include methyl, ethyl, propyl, and —CH(CH₃)₂.

The phrase “substituted alkyl” refers to an unsubstituted alkyl group as defined above in which one or more bonds to a carbon(s) or hydrogen(s) are replaced by a bond to non-hydrogen and non-carbon atoms such as, but not limited to, a halogen atom in halides such as F, Cl, Br, and I; an oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as in trialkylsilyl groups, dialkylarylsilyl groups, alkyldiarylsilyl groups, and triarylsilyl groups; and other heteroatoms in various other groups. Substituted alkyl groups also include groups in which one or more bonds to a carbon(s) or hydrogen(s) atom is replaced by a bond to a heteroatom such as oxygen in carbonyl, carboxyl, and ester groups; nitrogen in groups such as imines, oximes, hydrazones, and nitriles. Typical substituted alkyl groups include, among others, alkyl groups in which one or more bonds to a carbon or hydrogen atom is/are replaced by one or more bonds to fluorine atoms. One example of a substituted alkyl group is the trifluoromethyl group and other alkyl groups that contain the trifluoromethyl group. Other alkyl groups include those in which one or more bonds to a carbon or hydrogen atom is replaced by a bond to an oxygen atom such that the substituted alkyl group contains a hydroxyl, alkoxy, aryloxy group, or heterocyclyloxy group. Still other alkyl groups include alkyl groups that have an amine, alkylamine, dialkylamine, arylamine, (alkyl)(aryl)amine, diarylamine, heterocyclylamine, (alkyl)heterocyclyl)amine, (aryl)(heterocyclyl)amine, or diheterocyclylamine group.

The phrase “unsubstituted aryl” refers to aryl groups that do not contain heteroatoms. Thus the phrase includes, but is not limited to, groups such as phenyl, biphenyl, anthracenyl, naphthenyl by way of example. Although the phrase “unsubstituted aryl” includes groups containing condensed rings such as naphthalene, it does not include aryl groups that have other groups such as alkyl or halo groups bonded to one of the ring members, as aryl groups such as tolyl are considered herein to be substituted aryl groups as described below. A typical unsubstituted aryl group is phenyl. Unsubstituted aryl groups may be bonded to one or more carbon atom(s), oxygen atom(s), nitrogen atom(s), and/or sulfur atom(s) in the parent compound, however.

The phrase “substituted aryl group” has the same meaning with respect to unsubstituted aryl groups that substituted alkyl groups had with respect to unsubstituted alkyl groups. However, a substituted aryl group also includes aryl groups in which one of the aromatic carbons is bonded to one of the non-carbon or non-hydrogen atoms described above and also includes aryl groups in which one or more aromatic carbons of the aryl group is bonded to a substituted and/or unsubstituted alkyl, alkenyl, or alkynyl group as defined herein. This includes bonding arrangements in which two carbon atoms of an aryl group are bonded to two atoms of an alkyl, alkenyl, or alkynyl group to define a fused ring system (e.g. dihydronaphthyl or tetrahydronaphthyl). Thus, the phrase “substituted aryl” includes, but is not limited to tolyl, and hydroxyphenyl among others.

The phrase “unsubstituted alkenyl” refers to straight and branched chain and cyclic groups such as those described with respect to unsubstituted alkyl groups as defined above, except that at least one double bond exists between two carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The phrase “substituted alkenyl” has the same meaning with respect to unsubstituted alkenyl groups that substituted alkyl groups had with respect to unsubstituted alkyl groups. A substituted alkenyl group includes alkenyl groups in which a non-carbon or non-hydrogen atom is bonded to a carbon double bonded to another carbon and those in which one of the non-carbon or non-hydrogen atoms is bonded to a carbon not involved in a double bond to another carbon. Typical unsubstituted alkenyl groups have form 2 to 20 carbons, and more typical such groups have from 2 to 10 carbons.

The phrase “unsubstituted alkynyl” refers to straight and branched chain groups such as those described with respect to unsubstituted alkyl groups as defined above, except that at least one triple bond exists between two carbon atoms. Examples include, but are not limited to —C≡CH, —C≡CCH₃, —C≡CCH₂CH₃, —CH₂C≡CH, —CH₂C≡CCH₃, and —CH₂C≡CCH₂CH₃ among others. Typical unsubstituted alkynyl groups have form 2 to 20 carbons, and more typical groups have from 2 to 10 carbons.

The phrase “substituted alkynyl” has the same meaning with respect to unsubstituted alkynyl groups that substituted alkyl groups had with respect to unsubstituted alkyl groups. A substituted alkynyl group includes alkynyl groups in which a non-carbon or non-hydrogen atom is bonded to a carbon triple bonded to another carbon and those in which a non-carbon or non-hydrogen atom is bonded to a carbon not involved in a triple bond to another carbon.

The phrase “unsubstituted aralkyl” refers to unsubstituted alkyl groups as defined above in which a hydrogen or carbon bond of the unsubstituted allyl group is replaced with a bond to an aryl group as defined above. For example, methyl (—CH₃) is an unsubstituted alkyl group. If a hydrogen atom of the methyl group is replaced by a bond to a phenyl group, such as if the carbon of the methyl were bonded to a carbon of benzene, then the compound is an unsubstituted aralkyl group (i.e., a benzyl group). Thus the phrase includes, but is not limited to, groups such as benzyl, diphenylmethyl, and 1-phenylethyl (—CH(C₆H₅)(CH₃)) among others.

The phrase “substituted aralkyl” has the same meaning with respect to unsubstituted aralkyl groups that substituted aryl groups had with respect to unsubstituted aryl groups. However, a substituted aralkyl group also includes groups in which a carbon or hydrogen bond of the alkyl part of the group is replaced by a bond to a non-carbon or a non-hydrogen atom. Examples of substituted aralkyl groups include, but are not limited to, —CH₂C(═O)(C₆H₅), and —CH₂(2-methylphenyl) among others.

The phrase “unsubstituted heterocyclyl” refers to both aromatic and nonaromatic ring compounds including monocyclic, bicyclic, and polycyclic ring compounds such as, but not limited to, quinuclidyl, containing 3 or more ring members of which one or more is a heteroatom such as, but not limited to, N, O, and S. Although the phrase “unsubstituted heterocyclyl” includes condensed heterocyclic rings such as benzimidazolyl, it does not include heterocyclyl groups that have other groups such as alkyl or halo groups bonded to one of the ring members as compounds such as 2-methylbenzimidazolyl are substituted heterocyclyl groups. Examples of heterocyclyl groups include, but are not limited to: unsaturated 3 to 8 membered rings containing 1 to 4 nitrogen atoms such as, but not limited to pyrrolyl, pyrrolinyl, imidazolyl, pyrazolyl, pyridinyl, dihydropyridinyl, pyrimidyl, pyrazinyl, pyridazinyl, triazolyl (e.g. 4H-1,2,4-triazolyl, 1H-1,2,3-triazolyl, 2H-1,2,3-triazolyl etc.), tetrazolyl, (e.g. 1H-tetrazolyl, 2H-tetrazolyl, etc.); saturated 3 to 8 membered rings containing 1 to 4 nitrogen atoms such as, but not limited to, pyrrolidinyl, imidazolidinyl, piperidinyl, piperazinyl; condensed unsaturated heterocyclic groups containing 1 to 4 nitrogen atoms such as, but not limited to, indolyl, isoindolyl, indolinyl, indolizinyl, benzimidazolyl, quinolyl, isoquinolyl, indazolyl, benzotriazolyl; unsaturated 3 to 8 membered rings containing 1 to 2 oxygen atoms and 1 to 3 nitrogen atoms such as, but not limited to, oxazolyl, isoxazolyl, oxadiazolyl (e.g. 1,2,4-oxadiazolyl, 1,3,4-oxadiazolyl, 1,2,5-oxadiazolyl, etc.); saturated 3 to 8 membered rings containing 1 to 2 oxygen atoms and 1 to 3 nitrogen atoms such as, but not limited to, morpholinyl; unsaturated condensed heterocyclic groups containing 1 to 2 oxygen atoms and 1 to 3 nitrogen atoms, for example, benzoxazolyl, benzoxadiazolyl, benzoxazinyl (e.g. 2H-1,4-benzoxazinyl etc.); unsaturated 3 to 8 membered rings containing 1 to 3 sulfur atoms and 1 to 3 nitrogen atoms such as, but not limited to, thiazolyl, isothiazolyl, thiadiazolyl (e.g. 1,2,3-thiadiazolyl, 1,2,4-thiadiazolyl, 1,3,4-thiadiazolyl, 1,2,5-thiadiazolyl, etc.); saturated 3 to 8 membered rings containing 1 to 2 sulfur atoms and 1 to 3 nitrogen atoms such as, but not limited to, thiazolodinyl; saturated and unsaturated 3 to 8 membered rings containing 1 to 2 sulfur atoms such as, but not limited to, thienyl, dihydrodithiinyl, dihydrodithionyl, tetrahydrothiophene, tetrahydrothiopyran; unsaturated condensed heterocyclic rings containing 1 to 2 sulfur atoms and 1 to 3 nitrogen atoms such as, but not limited to, benzothiazolyl, benzothiadiazolyl, benzothiazinyl (e.g. 2H-1,4-benzothiazinyl, etc.), dihydrobenzothiazinyl (e.g. 2H-3,4-dihydrobenzothiazinyl, etc.), unsaturated 3 to 8 membered rings containing oxygen atoms such as, but not limited to furyl; unsaturated condensed heterocyclic rings containing 1 to 2 oxygen atoms such as benzodioxolyl (e.g., 1,3-benzodioxoyl, etc.); unsaturated 3 to 8 membered rings containing an oxygen atom and 1 to 2 sulfur atoms such as, but not limited to, dihydrooxathiinyl; saturated 3 to 8 membered rings containing 1 to 2 oxygen atoms and 1 to 2 sulfur atoms such as 1,4-oxathiane; unsaturated condensed rings containing 1 to 2 sulfur atoms such as benzothienyl, benzodithiinyl; and unsaturated condensed heterocyclic rings containing an oxygen atom and 1 to 2 oxygen atoms such as benzoxathiinyl. Heterocyclyl group also include those described above in which one or more S atoms in the ring is double-bonded to one or two oxygen atoms (sulfoxides and sulfones). For example, heterocyclyl groups include tetrahydrothiophene oxide and tetrahydrothiophene 1,1-dioxide. Typical heterocyclyl groups contain 5 or 6 ring members. More typical heterocyclyl groups include morpholine, piperazine, piperidine, pyrrolidine, imidazole, pyrazole, 1,2,3-triazole, 1,2,4-triazole, tetrazole, thiophene, thiomorpholine, thiomorpholine in which the S atom of the thiomorpholine is bonded to one or more O atoms, pyrrole, pyridine homopiperazine, oxazolidin-2-one, pyrrolidin-2-one, oxazole, quinuclidine, thiazole, isoxazole, furan, and tetrahydrofuran.

The phrase “substituted heterocyclyl” refers to an unsubstituted heterocyclyl group as defined above in which one or more of the ring members is bonded to a non-hydrogen atom such as described above with respect to substituted alkyl groups and substituted aryl groups. Examples include, but are not limited to, 2-methylbenzimidazolyl, 5-methylbenzimidazolyl, 5-chlorobenzthiazolyl, 1-methyl piperazinyl, 2-phenoxy-thiophene, and 2-chloropyridinyl among others. In addition, substituted heterocyclyl groups also include heterocyclyl groups in which the bond to the non-hydrogen atom is a bond to a carbon atom that is part of a substituted and unsubstituted aryl, substituted and unsubstituted arylalkyl, or unsubstituted heterocyclyl group. Examples include but are not limited to 1-benzylpiperdinyl, 3-phenylhiomorpholinyl, 3-(pyrrolidin-1-yl)-pyrrolidinyl, and 4-(piperidin-1-yl)-piperidinyl.

The phrase “unsubstituted heterocyclylalkyl” refers to unsubstituted alkyl groups as defined above in which a hydrogen or carbon bond of the unsubstituted alkyl group is replaced with a bond to a heterocyclyl group as defined above. For example, methyl(—CH₃) is an unsubstituted alkyl group. If a hydrogen atom of the methyl group is replaced by a bond to a heterocyclyl group, such as if the carbon of the methyl were bonded to carbon 2 of pyridine (one of the carbons bonded to the N of the pyridine) or carbons 3 or 4 of the pyridine, then the compound is an unsubstituted heterocyclylalkyl group.

The phrase “substituted heterocyclylalkyl” has the same meaning with respect to unsubstituted heterocyclylalkyl groups that substituted aralkyl groups had with respect to unsubstituted aralkyl groups. However, a substituted heterocyclylalkyl group also includes groups in which a non-hydrogen atom is bonded to a heteroatom in the heterocyclyl group of the heterocyclylalkyl group such as, but not limited to, a nitrogen atom in the piperidine ring of a piperidinylalkyl group. In addition, a substituted heterocyclylalkyl group also includes groups in which a carbon bond or a hydrogen bond of the alkyl part of the group is replaced by a bond to a substituted and unsubstituted aryl or substituted and unsubstituted arylalkyl group. Examples include but are not limited to phenyl-(piperidin-1-yl)-methyl and phenyl-(morpholin-4-yl)-methyl.

The phrase “unsubstituted alkoxy” refers to a hydroxyl group (—OH) in which the bond to the hydrogen atom is replaced by a bond to a carbon atom of an otherwise unsubstituted alkyl group as defined above.

The phrase “substituted alkoxy” refers to a hydroxyl group (—OH) in which the bond to the hydrogen atom is replaced by a bond to a carbon atom of an otherwise substituted alkyl group as defined above.

The term “protected” with respect to hydroxyl groups, amine groups, carboxyl groups, and sulfhydryl groups refers to forms of these functionalities which are protected from undesirable reaction using a protecting group known to those skilled in the art such as those set forth in Protective Groups in Organic Synthesis, Greene, T. W.; Wuts, P. G. M., John Wiley & Sons, New York, (3rd Edition, 1999) and The Practice of Peptide Synthesis, Bodanszky, M. and Bodanszky, A., Springer-Verlag, New York, (1984), which can be added or removed using the procedures set forth therein. Examples of protected hydroxyl groups include, but are not limited to, silyl ethers such as those obtained by reaction of a hydroxyl group with a reagent such as, but not limited to, t-butyldimethyl-chlorosilane, trimethylchlorosilane, triisopropylchlorosilane, triethylchlorosilane; substituted methyl and ethyl ethers such as, but not limited to methoxymethyl ether, methythiomethyl ether, benzyloxymethyl ether, t-butoxymethyl ether, 2-methoxyethoxymethyl ether, tetrahydropyranyl ethers, 1-ethoxyethyl ether, allyl ether, benzyl ether; esters such as, but not limited to, benzoylformate, formate, acetate, trichloroacetate, and trifluoroacetate. Examples of protected amine groups include, but are not limited to, amides such as, formamide, acetamide, trifluoroacetamide, and benzamide; imides, such as phthalimide, and dithiosuccinimide; carbamates such as t-butyl carbamate (Boc), fluorenylmethyl carbamate (Fmoc), and benzyl carbamate (Cbz); and others. Examples of protected sulfhydryl groups include, but are not limited to, thioethers such as S-t-butyl thioether, S-benzyl thioether, and S-4-picolyl thioether; substituted S-methyl derivatives such as hemithio, dithio and aminothio acetals; and others. Examples of protected carboxyl groups include but are not limited to esters such as methyl, ethyl, t-butyl, trimethylsilylethyl, benzyl, and the like.

A “pharmaceutically acceptable salt” includes a salt with an inorganic base, organic base, inorganic acid, organic acid, or basic or acidic amino acid. As salts of inorganic bases, the invention includes, for example, alkali metals such as sodium or potassium; alkaline earth metals such as calcium and magnesium or aluminum; and ammonia. As salts of organic bases, the invention includes, for example, trimethylamine, triethylamine, pyridine, picoline, ethanolamine, diethanolamine, and triethanolamine. As salts of inorganic acids, the instant invention includes, for example, hydrochloric acid, hydroboric acid, nitric acid, sulfuric acid, and phosphoric acid. As salts of organic acids, the instant invention includes, for example, formic acid, acetic acid, trifluoroacetic acid, fumaric acid, oxalic acid, tartaric acid, maleic acid, citric acid, succinic acid, malic acid, methanesulfonic acid, benzenesulfonic acid, and p-toluenesulfonic acid. As salts of basic amino acids, the instant invention includes, for example, arginine, lysine and ornithine. Acidic amino acids include, for example, aspartic acid and glutamic acid.

Certain compounds within the scope of Formula I are derivatives referred to as prodrugs. The expression “prodrug” denotes a derivative of a known direct acting drug, e.g. esters and amides, which derivative has enhanced delivery characteristics and therapeutic value as compared to the drug, and is transformed into the active drug by an enzymatic or chemical process; see Notari, R. E., “Theory and Practice of Prodrig Kinetics,” Methods in Enzymology 112:309-323 (1985); Bodor, N., “Novel Approaches in Prodrug Design,” Drugs of the Future 6:165-182 (1981); and Bundgaard, H., “Design of Prodrugs: Bioreversible-Derivatives for Various Functional Groups and Chemical Entities,” in Design of Prodrugs (H. Bundgaard, ed.), Elsevier, New York (1985), Goodman and Gilmans, The Pharmacological Basis of Therapeutics, 8th ed., McGraw-Hill, Int. Ed. 1992. The preceding references are hereby incorporated by reference in their entirety.

Tautomers refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, ketones are typically in equilibrium with their enol forms. Thus, ketones and their enols are referred to as tautomers of each other. As readily understood by one skilled in the art, a wide variety of functional groups and other structures may exhibit tautomerism, and all tautomers of compounds having formula I are within the scope of the present invention.

Compounds of the present invention include enriched or resolved optical isomers at any or all asymmetric atoms as are apparent from the depictions. Both racemic and diastereomeric-mixtures, as well as the individual optical isomers can be isolated or synthesized so as to be substantially free of their enantiomeric or diastereomeric partners, and these are all within the scope of the invention.

“Treating” within the context of the instant invention, means an alleviation, in whole or in part, of symptoms associated with a disorder or disease, or halt of further progression or worsening of those symptoms, or prevention or prophylaxis of the disease or disorder. Similarly, as used herein, a “therapeutically effective amount” of a compound of the invention refers to an amount of the compound that alleviates, in whole or in part, symptoms associated with a disorder or disease, or halts of further progression or worsening of those symptoms, or prevents or provides prophylaxis for the disease or disorder. Treatment may also include administering the pharmaceutical formulations of the present invention in combination with other therapies. For example, the compounds of the invention can also be administered in conjunction with other anti-inflammatory agents or agents used for the treatment of metabolic disorders.

Compounds of formula I, where A is —NH—CR^{3a}R^{3b}— and Y is phenyl (e.g., formula II) may be readily prepared using the Hantzsch reaction. For example, a compound of formula II may readily be prepared as shown in Scheme 1. A substituted benzaldehyde, beta-keto ester, ammonium hydroxide, and a beta-keto ester substituted with a protected amine may be combined in a suitable solvent such as ethanol and exposed to heat to give the dihydropyridine. The reaction can be heated by microwave radiation to shorten the reaction times. The dihydropyridine is oxidized using a suitable agent such as DDQ, CAN or [bis(trifluoroacetoxy)iodo]benzene in a solvent, such as methylene chloride, to give the pyridine. The protected amine is deprotected to allow closure of the 5-member ring. Suitable protecting groups include acid-labile groups such as t-butyloxycarbonyl that may be removed using HCl or trifluoroacetic acid. When acidolysis is performed to remove the protecting group, the resulting ammonium salt must be neutralized with a base such as LiOH, pyridine or diisopropylethylamine for ring closure to occur. The N-oxide may be prepared by oxidizing the pyridine nitrogen with m-chloroperbenzoic acid [see Deady, Synth. Comm. 1977, 509-14], H₂O₂ [Donnici et al. J. Braz. Chem. Soc. 1998, 5:455-60] or another suitable agent. Those of skill in the art will appreciate that similar methodology can be used to readily prepare other compounds of formula I, where A is —NH—CR^{3a}R^{3b}— and Y is other aryl, or heteroaryl. Literature methods may also be modified to access compounds where Y is cycloalkyl as well as nonaromatic heterocyclyl [see, e.g., J. Chem. Soc. Perkin Trans., 1999, 1755; Synthesis, 2000, 1532; J. Org. Chem., 1997, 62:3582].
Scheme 1

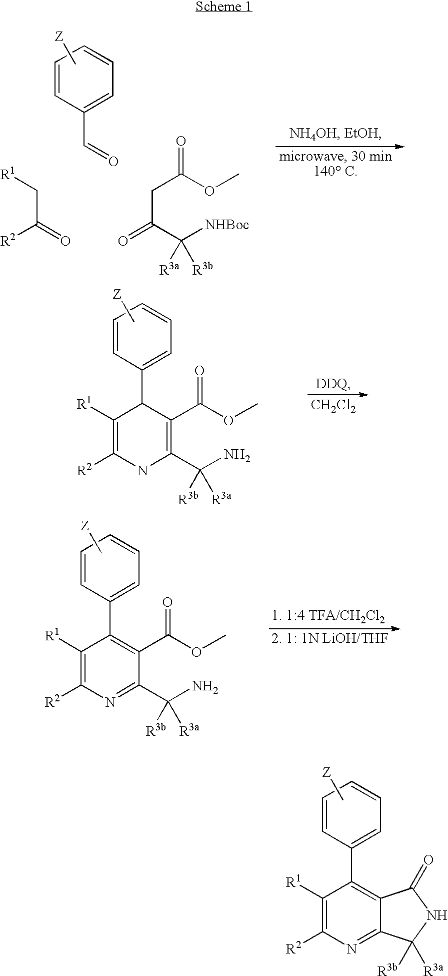

Alternatively, compounds of formula I where A is —NH—CR^{3a}R^{3b}— and Y is phenyl (e.g., formula II) may also be prepared as shown in Scheme 2. A substituted benzaldehyde, substituted enamine, ammonium hydroxide, and a tetramic acid may be combined in a suitable solvent such as ethanol and exposed to heat to give the dihydropyridine. The latter compound may be oxidized to the pyridine with CAN under acidic conditions (e.g., TFA) or other suitable oxidant as disclosed herein. Similarly, compounds of formula I where A is —NH—CR^{3a}R^{3b}— and Y is other aryl, heterocyclyl or cycloalkyl may be made by modification of the present methods or related literature methods.
Scheme 2

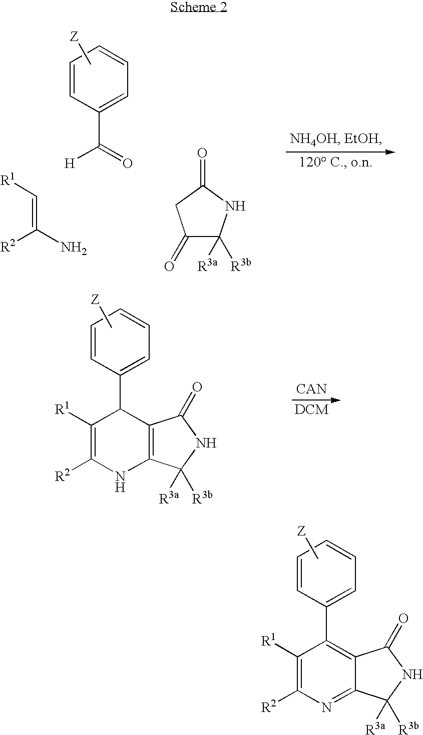

In addition, compounds of the invention may be prepared in a step-wise fashion on solid phase as shown in Scheme 3. While any suitable solid phase may be used, polystyrene resins having a free hydroxyl, e.g., WANG resin, are convenient. The beta-keto ester containing a protected amine may be formed on the resin by reaction of a derivative of Meldrum's acid with the resin-bound hydroxyl in the first step. Reaction of the resulting beta keto-ester with a benzaldehyde derivative provides the beta-keto alpha-olefin ester of the second step. Subsequent reaction with an enamine ester provides the dihydropyridine which may be oxidized to the pyridine as described above. Ring closure and release from the resin may be effected using the same acidolysis/neutralization steps as described above. Alternatively, other protecting groups on the molecule such as allyl ester may be removed as shown and standard amide coupling techniques may be used to install additional amide moieties. The reactions may be conducted in any solvent that both swells the resin and solubilizes the reactants; typically DMF is used. While the scheme illustrates the synthesis of a compound of Formula If, the methods are not so limited. Those of skill in the art will readily understand that other compounds having formula I may be prepared by slight modification of the illustrated methods.
Scheme 3

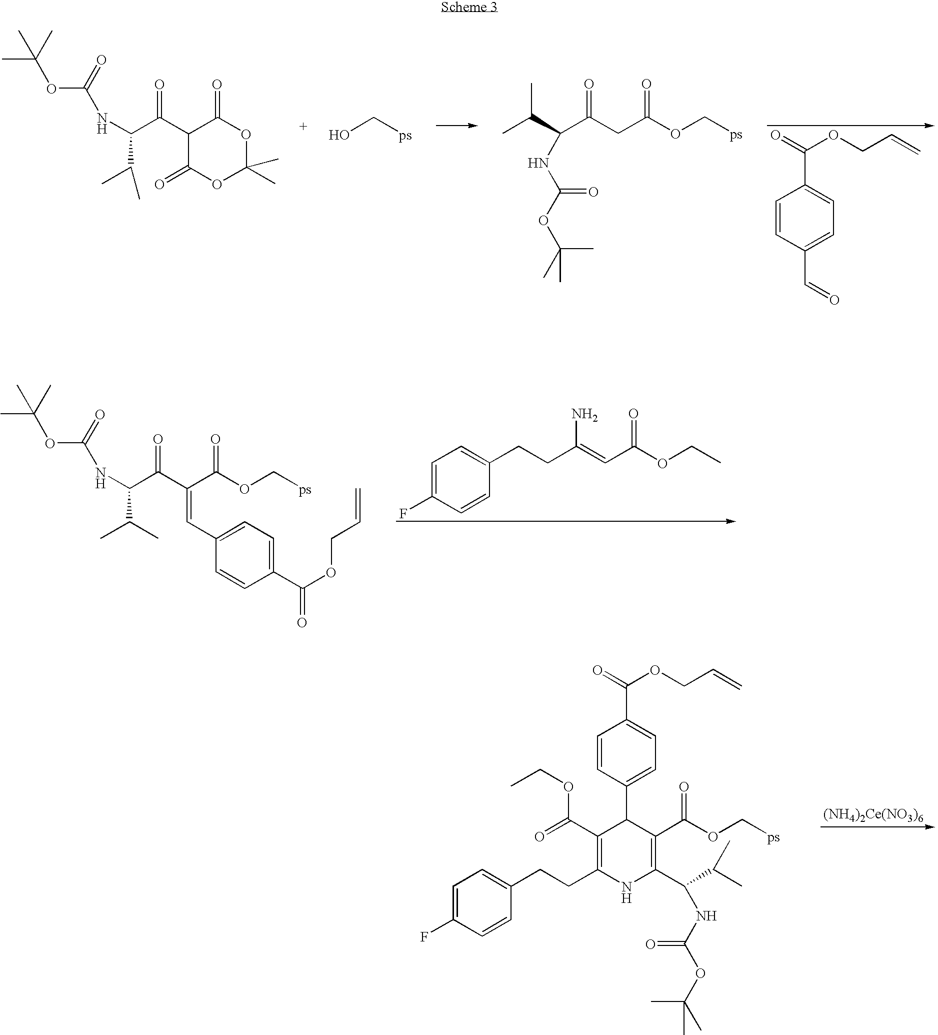

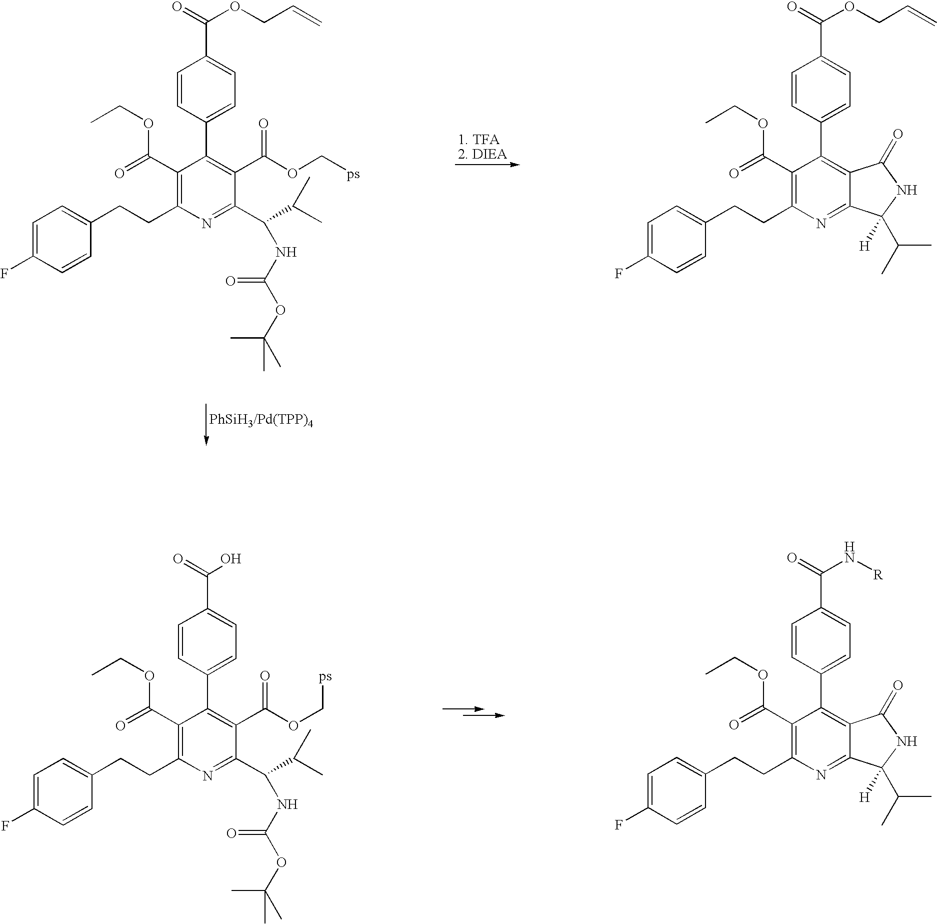

Compounds of formula I where A is —CRR′—CR^{4a}R^{4b}—CRR′—, Y is phenyl, and R and R′ are both H (e.g., formula III) may also be prepared as shown in Scheme 4. A substituted benzaldehyde, substituted enamine, ammonium hydroxide, and a cyclohexene-1,3-dione may be combined in a suitable solvent such as ethanol and exposed to heat to give the dihydropyridine. The latter compound may be oxidized to the pyridine with CAN under acidic conditions (e.g., TFA) or another suitable oxidant. Those of skill in the art will readily appreciate that other compounds of Formula I where Y is other aryl, heterocyclyl, or cycloalkyl groups may be made as described above for compounds where A is —NH—CR^{3a}R^{3b}—.
Scheme 4

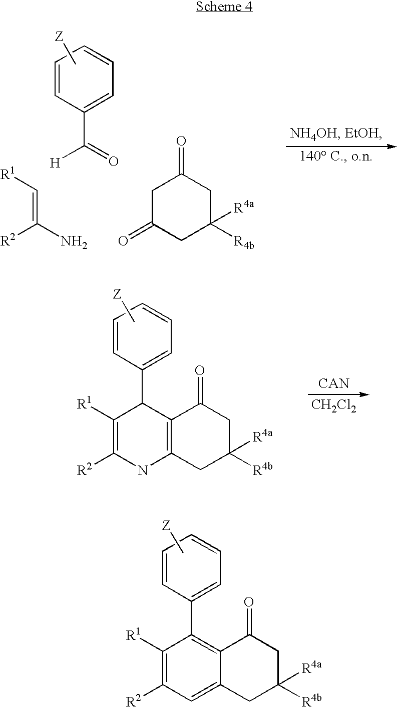

The instant invention also provides for pharmaceutical compositions which may be prepared by mixing one or more compounds of the invention, pharmaceutically acceptable salts thereof, stereoisomers thereof, tautomers thereof, or solvates thereof, with pharmaceutically acceptable carriers, excipients, binders, diluents or the like to treat or ameliorate a variety of disorders mediated by calcitonin and/or amylin receptors. The compositions of the invention may be used to create formulations and prevent or treat disorders mediated by calcitonin and/or amylin receptors such as bone and metabolic diseases. Such compositions can be in the form of, for example, granules, powders, tablets, capsules, syrup, suppositories, injections, emulsions, elixirs, suspensions or solutions. The instant compositions can be formulated for various routes of administration, for example, by oral administration, by nasal administration, by rectal administration, subcutaneous injection, intravenous injection, intramuscular injections, or intraperitoneal injection. The following dosage forms are given by way of example and should not be construed as limiting the instant invention.

For oral, buccal, and sublingual administration, powders, suspensions, granules, tablets, pills, capsules, gelcaps, and caplets are acceptable as solid dosage forms. These can be prepared, for example, by mixing one or more compounds of the instant invention, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

For nasal administration, the pharmaceutical formulations and medicaments may be a spray or aerosol containing an appropriate solvent(s) and optionally other compounds such as, but not limited to, stabilizers, antimicrobial agents, antioxidants, pH modifiers, surfactants, bioavailability modifiers and combinations of these. A propellant for an aerosol formulation may include compressed air, nitrogen, carbon dioxide, or a hydrocarbon based low boiling solvent.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Preferably, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

For rectal administration, the pharmaceutical formulations and medicaments may be in the form of a suppository, an ointment, an enema, a tablet or a cream for release of compound in the intestines, sigmoid flexure and/or rectum. Rectal suppositories are prepared by mixing one or more compounds of the instant invention, or pharmaceutically acceptable salts or tautomers of the compound, with acceptable vehicles, for example, cocoa butter or polyethylene glycol, which is present in a solid phase at normal storing temperatures, and present in a liquid phase at those temperatures suitable to release a drug inside the body, such as in the rectum. Oils may also be employed in the preparation of formulations of the soft gelatin type and suppositories. Water, saline, aqueous dextrose and related sugar solutions, and glycerols may be employed in the preparation of suspension formulations which may also contain suspending agents such as pectins, carbomers, methyl cellulose, hydroxypropyl cellulose or carboxymethyl cellulose, as well as buffers and preservatives.

Besides those representative dosage forms described above, pharmaceutically acceptable excipients and carriers are generally known to those skilled in the art and are thus included in the instant invention. Such excipients and carriers are described, for example, in “Remingtons Pharmaceutical Sciences” Mack Pub. Co., New Jersey (1991), which is incorporated herein by reference.

The formulations of the invention may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant invention.

A therapeutically effective amount of a compound of the present invention may vary depending upon the route of administration and dosage form. The typical compound or compounds of the instant invention is a formulation that exhibits a high therapeutic index. The therapeutic index is the dose ratio between toxic and therapeutic effects which can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present invention, thus generally described, will be understood more readily by reference to the following examples, which are provided by way of illustration and are not intended to be limiting of the present invention.

### EXAMPLES

The following abbreviations are used throughout the application:

| | |
|---|---|
| AcOH | Acetic acid |
| CAN | Ceric (IV) ammonium nitrate |
| DCM | Dichloromethane |
| DDQ | 2,3-Dichloro-5,6-Dicyano-1,4-benzoquinone |
| DMAP | 4-N,N-dimethylaminopyridine |
| EDCI | 1-[3-(Dimethylamino)propyl]-3-ethyl-carbodiimide |
| EtOH | Ethanol |
| EtOAc | Ethyl acetate |
| LCMS | Liquid chromatography mass spectroscopy |
| MeOH | Methanol |
| PhMe | Toluene |
| r.t. | Room temperature |

### Example 1

### Synthesis of Ethyl 3-amino-5-(4-fluorophenyl)-2-pentenoate, (4)

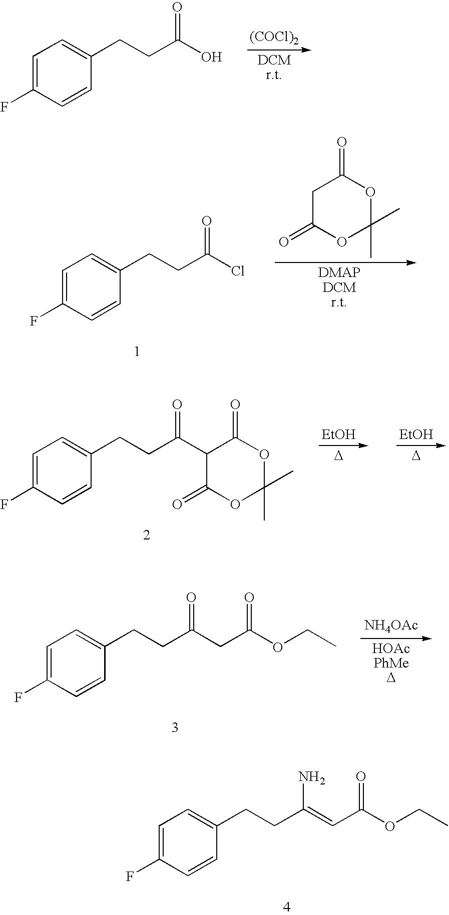

Oxalyl chloride (31 mL, 357 mmol) was added to a solution of 3-(4-fluorophenyl)propionic acid (20 g, 119 mmol) in dichloromethane (100 mL) and the mixture was stirred overnight at r.t. After concentrating in vacuo and drying in high vacuum, the crude acid chloride 1 was added dropwise to a cooled solution of Meldrum's acid (18.86 g, 131 mmol, 1.1 eq) and 4-N,N-dimethylaminopyridine (DMAP, 15.99 g, 131 mmol, 1.1 eq) in dichloromethane (100 mL) at 0° C. and the resulting mixture was stirred overnight at r.t. The solvent was evaporated in vacuo, ethyl acetate (150 mL) was added and the solution was washed with water (150 mL), 1 M aqueous HCl (3×150 mL) and brine (150 mL). The organic layer was dried over Na₂SO₄, concentrated in vacuo and dried in high vacuum to give crude compound 2 as an oil Which was dissolved in ethanol (200 mL). The solution was heated to reflux overnight. After concentrating in vacuo and drying in high vacuum, the crude compound 3 was added to a mixture of ammonium acetate (45.86 g, 595 mmol), acetic acid (6.9 mL, 119 mmol) and toluene (220 mL). After heating to reflux overnight with azeotropic removal of water, the solvent was removed in vacuo. Silica gel column chromatography (hexane −20% ethyl acetate/hexane) yielded the title compound (7.7 g, 27% yield for 4 steps) as a pale yellow oil. ¹H NMR (400 MHz, CDCl₃): δ 1.28 (t, 3H), 2.41 (t, 2H), 2.86 (t, 2H), 4.13 (q, 2H), 4.58 (s, 1H), 7.00 (m, 2H), 7.16 (m, 2H).

### Example 2

### Synthesis of 4-Formyl-N-furan-2-ylmethylbenzamide, (6)

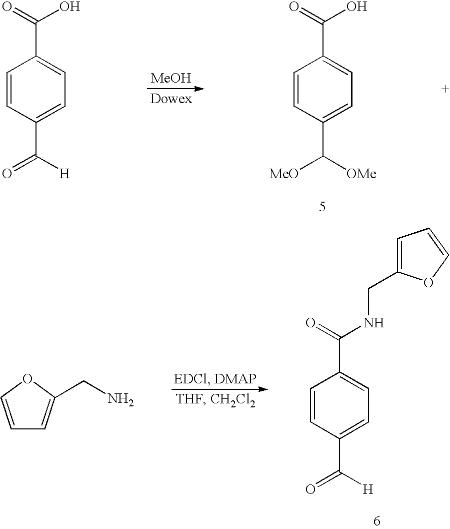

### Step 1: 4-(Dimethoxymethyl)benzoic acid, (5)

To a solution of 4-carboxybenzaldehyde (11.26 g, 75.0 mmol) in MeOH (200 mL) was added Dowex 50×8-100 ion exchange resin (2.25 g). The mixture was stirred at reflux for 16 hours. The resin was removed by vacuum filtration and the solvent was then removed by rotary evaporation. The white solid remaining was triturated with hot hexane, was filtered, and was dried in vacuo, yielding the title compound as a white powder (12.36 g, 84% yield). ¹H NMR (400 MHz, CDCl₃): δ 8.03 (d, J=8.3 Hz, 2H), 7.54 (d, J=8.3 Hz, 2H), 5.45 (s, 1H), 3.34 (s, 6H).

### Step 2: 4-Formyl-N-furan-2-ylmethylbenzamide, (6)

A solution of 4-(dimethoxymethyl)benzoic acid 5 (2.94 g, 15 mmol), EDCI (5.75 g, 30 mmol), furfurylamine (3.5 mL, 37.5 mmol), and DMAP (122 mg, 1 mmol) in THF (200 mL) and CH₂Cl₂ (50 mL) was stirred at r.t. for 16 h. The solvent was removed by rotary evaporation and the resulting yellow oil was dissolved in CHCl₃ and was washed twice with 0.5 M HCl. The organic solution was then vigorously stirred with 1.0 M HCl for 2 h. The layers were separated and the organic phase was washed twice more with 1.0 M HCl and once with brine. The organic phase was dried over Na₂SO₄ and the solvent was removed by rotary evaporation. The crude product was crystallized from hot EtOAc, yielding the title compound as a pale yellow solid in 2 crops (2.09 g, 58% yield). ¹H NMR (400 MHz, CDCl₃): δ 10.09 (s, 1H), 7.97 (s, 4H), 7.42 (m, 1H), 6.54 (br s, 1H), 6.39 (dd, J=2.0, 3.3 Hz, 1H), 6.35 (d, J=3.3 Hz, 1H), 4.69 (d, J=5.3 Hz, 2H).

### Example 3

### Synthesis of Tetramic Acid, (9)—

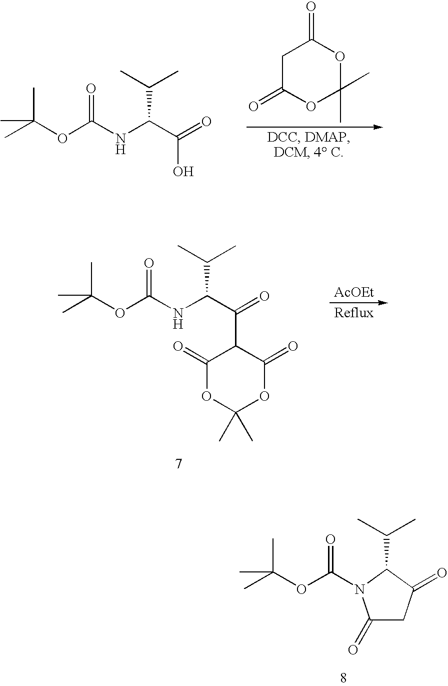

### Step 1: [(R)-1-(2,2-Dimethyl-4,6-dioxo-[1,3]dioxane-5-carbonyl)-2-methyl-propyl]-carbamic acid tert-butyl ester (7)

N-Boc protected Valine (1.09 g, 5 mmol,) was mixed at 0° C. with Meldrum's acid (2,2-dimethyl-1,3-dioxane-4,6-dione; 0.72 g, 5 mmol), dicyclohexylcarbodiimide (DCC; 1.08 g, 5 mmol, 1.0) and 4-dimethylaminopyridine (DMAP, 0.64 g, 5.25 mmol) in CH₂Cl₂. The reaction mixture was kept at 4° C. overnight (16 h). After that, precipitated dicyclohexyl urea was removed by vacuum filtration and the solution was washed 4× with 5% aq. solution of potassium bisulfate and brine. The organic phase was than dried with MgSO₄ at 4° C. for 1 hour. Crude product (7, 1.8 g, 100%) obtained after evaporation was used for next reaction without purification.

### Step 2: 1-Butoxycarbonyl-2-Isopropyl-3,5-dioxo-pyrrolidine, (8)

Compound 7 was dissolved in EtOAc and refluxed for 3 hr. and evaporated to 1.3 g of amorphous solid. Silica gel column chromatography (20% ethyl acetate/hexane-ethyl acetate) yielded the title compound (0.97 g, 80% yield for 2 steps) as a yellow oil.

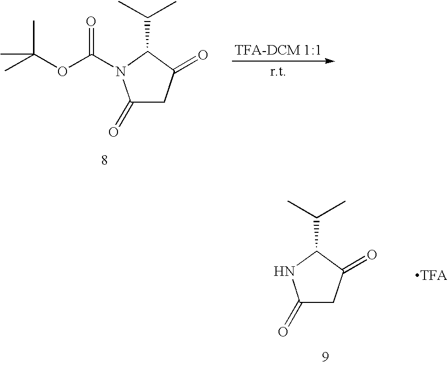

### Step 3: (R)-5-Isopropyl-pyrrolidine-2,4-dione (9)

Compound 8 (0.90 mg, 3.73 mmol) was dissolved in 50% TFA/CH₂Cl₂ and after 30 minutes was evaporated to dryness to give the title compound (0.99 mg, quantitative, contains one equivalent of TFA) as a pale yellow oil which crystallized upon standing. ¹H NMR (400 MHz, CDCl₃): δ 0.97 (d, 3H), 1.08 (d, 3H), 2.23 (m, 1H), 3.11 (d, 1H), 3.16 (d, 1H), 3.97 (d, 1H), 8.16 (br. s, 1H).

### Example 4

### Dihydropyridine and Pyridine Formation

Method A

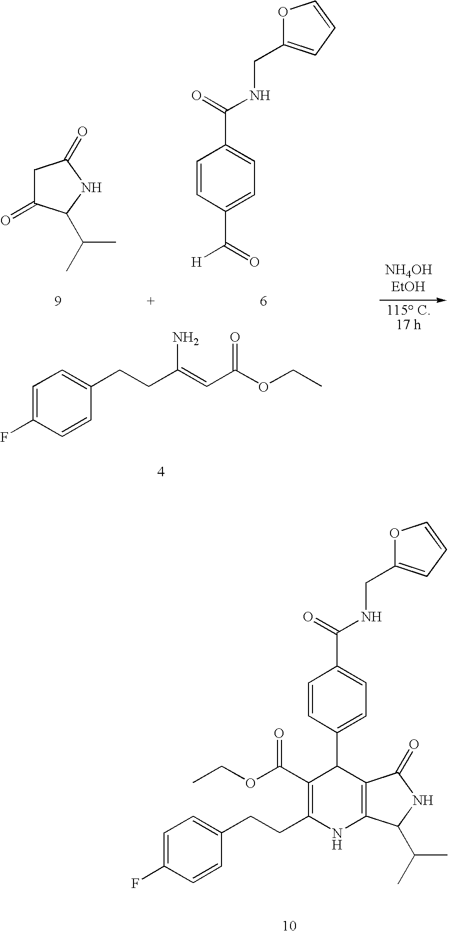

### Step 1: 2-[2-(4-Fluoro-phenyl)-ethyl]-4-{4-[(furan-2-ylmethyl)-carbamoyl]-phenyl}-7-isopropyl-5-oxo-4,5,6,7-tetrahydro-1H-pyrrolo[3,4-b]pyridine-3-carboxylic acid ethyl ester (10)

Aqueous ammonia (0.15 mL) was added to a solution of compounds 9 (264 mg, 1.87 mmol, 3 eq), 4 (192 mg, 0.809 mmol, 1.3 eq), and 6 (143 mg, 0.623 mmol, 1 eq) in ethanol (2.7 mL). The mixture was heated in a closed vial under argon to 115° C. for 17 h. After cooling down, the solvent was concentrated in vacuo and the crude product was purified by silica gel column chromatography (dichloromethane −10% methanol/dichloromethane) yielding the title compound (77 mg, 22% yield) as a mixture of two diastereoisomers.

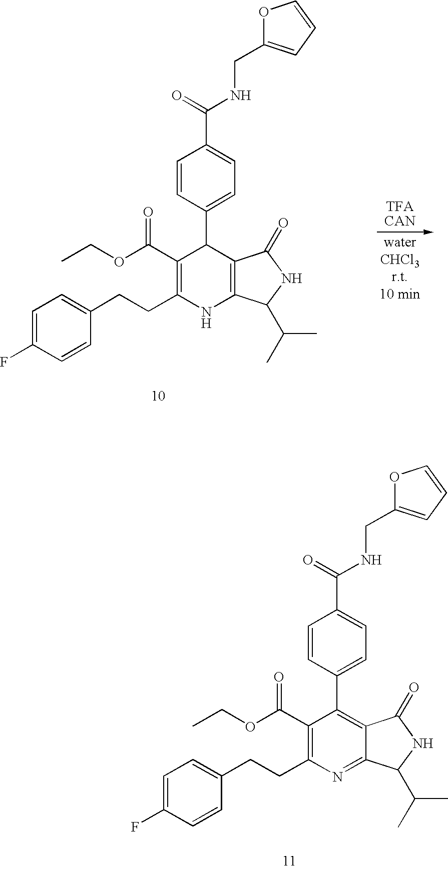

### Step: 2-[2-(4-Fluoro-phenyl)-ethyl]-4-{4-[(furan-2-ylmethyl)-carbamoyl]-phenyl}-7-isopropyl-5-oxo-6,7-dihydro-5H-pyrrolo[3,4-b]pyridine-3-carboxylic acid ethyl ester (11)

An aqueous solution of CAN (1 M, 1 mL) was added to a solution of compound 10 (77 mg, 0.135 mmol) and trifluoroacetic acid (0.1 mL) in CHCl₃ (35 mL). The resulting mixture was vigorously stirred for 10 min at r.t. The organic layer was separated, washed with water, dried over Na₂SO₄, and concentrated in vacuo to yield the crude product (82 mg, 80% purity by LCMS). Purification by preparative LCMS (PE SCIEX, C₁₈, acetonitrile-water with 0.035% trifluoroacetic acid) yielded the title compound (36.5 mg, 98% purity by LCMS, 40% yield) as a pale yellow oil. Product can be recrystallized from an acetonitrile/water mixture yielding more than 99.9% pure material. ¹H NMR (400 MHz, CDCl₃): δ 0.73 (d, 3H), 0.95 (t, 3H), 1.13 (d, 3H), 2.50 (m, 1H), 3.13 (m, 2H), 3.23 (m, 2H), 4.02 (q, 2H), 4.54 (d, 1H), 4.65 (d, 2H), 6.32 (m, 1H), 6.36 (m, 1H), 6.71 (br. t, 1H), 6.97 (m, 2H), 7.17 (m, 2H), 7.34 (br. s, 1H), 7.39 (m, 1H), 7.44 (d, 2H), 7.83 (d, 2H).

### Example 5

### Synthesis of 1-[4-(4-Fluoro-phenyl)-2-oxo-butyl]-pyridinium trifluoroacetate

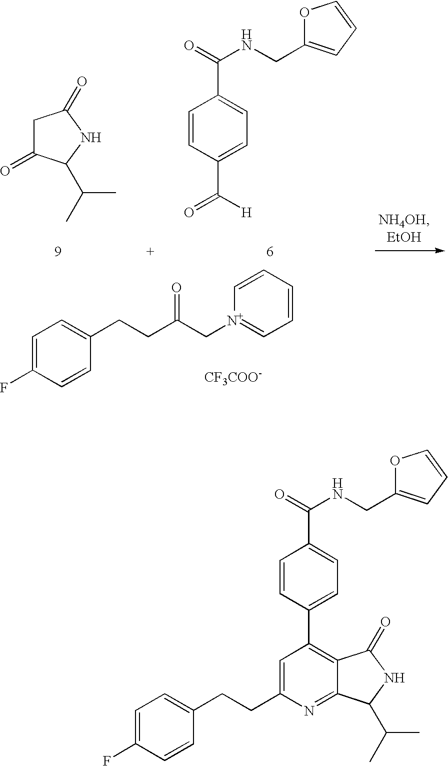

### Step 1: 4-{2-[2-(4-Fluoro-phenyl)-ethyl]-7-isopropyl-5-oxo-6,7-dihydro-5H-pyrrolo[3,4-b]pyridin-4-yl}-N-furan-2-ylmethyl-benzamide

1-[4-(4-Fluoro-phenyl)-2-oxo-butyl]-pyridinium trifluoroacetate (214 mg, 0.6 mmol) and 7 (113 mg, 0.8 mmol) were dissolved in EtOH (1.3 mL) containing 100 μL NH₄OH. Aldehyde 6 (92 mg, 0.4 mmol) was added and closed vial was stirred at 120° C. for 22 hrs. The mixture was purified by preparative LCMS (PE SCIEX, C₁₈, acetonitrile-water with 0.035% trifluoroacetic acid), followed by column chromatography (silica gel, 0-10% MeOH/DCM), yielding the title compound in 4.9 mg yield.

### Example 6

### Synthesis of 2-[2-(4-Fluoro-phenyl)-ethyl]-4-{4-[(furan-2-ylmethyl)-carbamoyl]-phenyl}-7,7-dimethyl-5-oxo-5,6,7,8-tetrahydro-quinoline-3-carboxylic acid ethyl ester

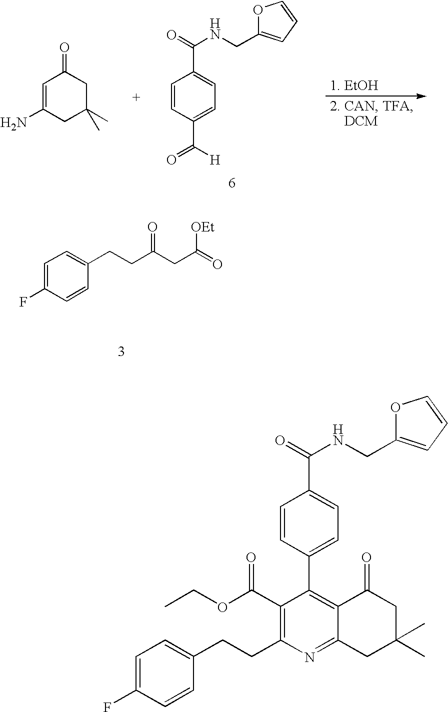

Step 1: Compounds 3 (66 mg, 0.280 mmol), 6 (50 mg, 0.218 mmol) and 3-amino-5,5-dimethyl-cyclohex-2-enone (32 mg, 0.23 mmol) were dissolved in 1 mL EtOH and stirred at 120° C. overnight. The solvent was evaporated and the residue was purified by silica gel chromatography (0-5% MeOH/DCM) to yield 80 mg of 2-[2-(4-fluoro-phenyl)-ethyl]-4-{4-[(furan-2-ylmethyl)-carbamoyl]-phenyl}-7,7-dimethyl-5-oxo-1,4,5,6,7,8-hexahydro-quinoline-3-carboxylic acid ethyl ester.

Step 2: An aqueous solution of ammonium cerium (IV) nitrate (CAN, 0.5M, 1 mL) was added to a solution of the compound obtained in the previous reaction (80 mg, 0.14 mmol) and trifluoroacetic acid (25 μL) in DCM (6 mL). The resulting mixture was vigorously stirred for 10 min at r.t. The organic layer was separated, washed with water, dried over MgSO₄, and concentrated in vacuo to yield the crude product. Purification by preparative LCMS (PE SCIEX, C₁₈, acetonitrile-water with 0.035% trifluoroacetic acid) yielded the title compound (70 mg)

### Example 7

Calcitonin binding assay. Competition binding assays are carried out using the T47D cell line. T47D cells are a human breast cancer cell line that expresses functional calcitonin receptors. Cells are plated at 5×10⁴ cells/well in 96-well plates and cultured overnight at 37° C. in 5% CO₂. Culture media is aspirated, the cells washed in serum-free culture medium containing 0.1% (w/v) bovine serum albumin, and incubated with ¹²⁵I-labeled salmon calcitonin (100,000 cpm; 80-120 pmol/liter; Amersham Biosciences, Piscataway, N.J.) and appropriate concentrations of test or reference compounds. Non-specific binding of ¹²⁵I-labeled calcitonin is assessed using 300 nM unlabelled salmon calcitonin. After incubation for 1 hr at 37° C. the medium is aspirated and the cells washed with 4° C. phosphate-buffered saline. Cells and bound radioactivity are recovered from the wells using 0.5 M NaOH and counted using a Packard automatic γ-scintillation spectrometer.

Calcitonin cAMP activity assay. The functional assay for calcitonin agonists is carried out with intact T47D cells using accumulation of cAMP as readout. T47D cells are plated at 5×10³ cells/well in 96-well plates and cultured overnight at 37° C. in 5% CO₂. Culture media is aspirated, replaced with 50 μL/well of serum-free culture medium containing 20 mM HEPES (pH 7.4) and 0.1% (w/v) bovine serum albumin (Assay Medium). After incubation for 10 min at 37° C., 50 μL of Assay Medium containing 1 mM isobutylmethylxanthine and appropriate concentrations of test or reference compounds is added. The cells are incubated for a further 30 min, the media aspirated and the cells lysed in 40 μL/well of phosphate-buffered saline containing 0.5% Triton X-100. The amount of cAMP in 10 μL of lysate is measured using a commercially available homogenous time-resolved fluorescent assay (CIS-US Inc., Bedford Mass.).

### Example 8

Amylin cAMP activity assay. The functional assay for amylin agonists follows the general methodologies to assess the functional activities of the calcitonin receptor vs. the amylin receptor using transient expression techniques, as described in Christopoulos G, Perry K J, Morfis M, Tilakaratne N, Gao Y, Fraser N J, Main M J, Foord S M, Sexton P M., Mol Pharmacol. 1999 July; 56(1):235-42. (Multiple amylin receptors arise from receptor activity-modifying protein interaction with the calcitonin receptor gene product.)

### Example 9

Compounds listed in the following Table were synthesized and assayed or will be assayed using the above procedures. Compound identity was verified by mass spectroscopy, with all molecules showing the expected mass ion. All compounds in the following Table exhibited activity in either the calcitonin or amylin cAMP functional assay and/or are expected to exhibit activity in the amylin cAMP functional assay.

TABLE

| Compound | | | Calc. |
|---|---|---|---|
| Number | Compound Structure | Compound Name | MW |
| 12 | 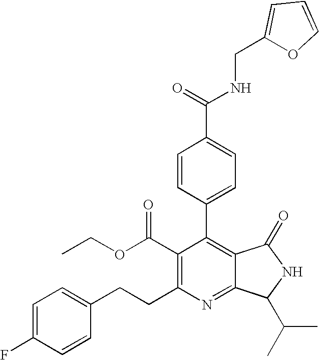 | 2-[2-(4-Fluoro-phenyl)-ethyl]-4-{4- [(furan-2-ylmethyl)-carbamoyl]-phenyl}- 7-isopropyl-5-oxo-6,7-dihydro-5H- pyrrolo[3,4-b]pyridine-3-carboxylic acid ethyl ester | 570 |
| 13 | 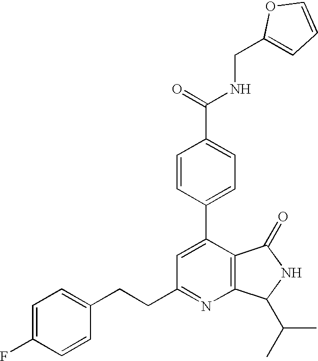 | 4-{2-[2-(4-Fluoro-phenyl)-ethyl]-7- isopropyl-5-oxo-6,7-dihydro-5H- pyrrolo[3,4-b]pyridin-4-yl}-N-furan-2- ylmethyl-benzamide | 498 |
| 14 | 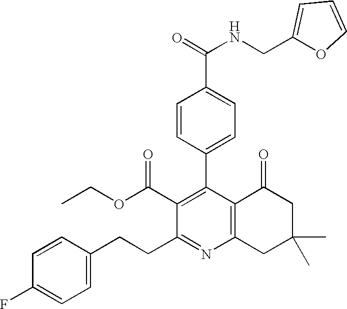 | 2-[2-(4-Fluoro-phenyl)-ethyl]-4-{4- [(furan-2-ylmethyl)-carbamoyl]-phenyl}- 7,7-dimethyl-5-oxo-5,6,7,8-tetrahydro- quinoline-3-carboxylic acid ethyl ester | 569 |
| 15 | 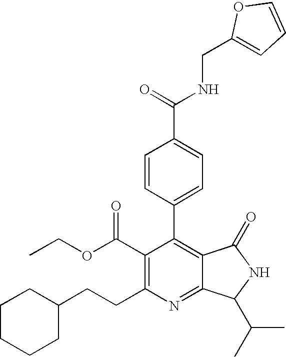 | 2-(2-Cyclohexyl-ethyl)-4-{4-[(furan-2- ylmethyl)-carbamoyl]-phenyl}-7- isopropyl-5-oxo-6,7-dihydro-5H- pyrrolo[3,4-b]pyridine-3-carboxylic acid ethyl ester | 558 |
| 16 | 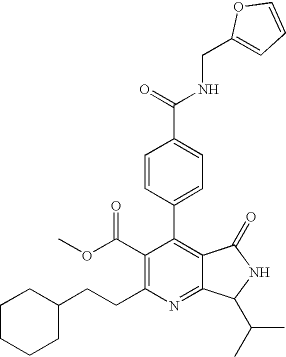 | 2-(2-Cyclohexyl-ethyl)-4-{4-[(furan-2- ylmethyl)-carbamoyl]-phenyl}-7- isopropyl-5-oxo-6,7-dihydro-5H- pyrrolo[3,4-b]pyridine-3-carboxylic acid methyl ester | 544 |
| 17 | 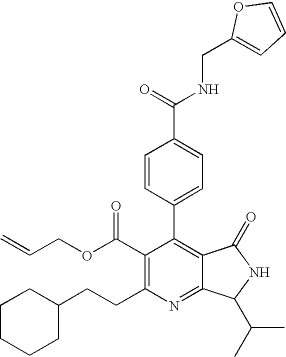 | 2-(2-Cyclohexyl-ethyl)-4-{4-[(furan-2- ylmethyl)-carbamoyl]-phenyl}-7- isopropyl-5-oxo-6,7-dihydro-5H- pyrrolo[3,4-b]pyridine-3-carboxylic acid allyl ester | 570 |
| 18 | 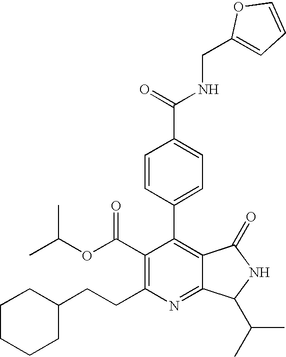 | 2-(2-Cyclohexyl-ethyl)-4-{4-[(furan-2- ylmethyl)-carbamoyl]-phenyl}-7- isopropyl-5-oxo-6,7-dihydro-5H- pyrrolo[3,4-b]pyridine-3-carboxylic acid isopropyl ester | 572 |
| 19 | 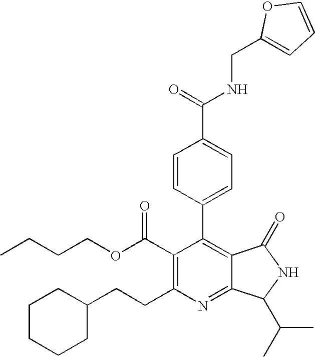 | 2-(2-Cyclohexyl-ethyl)-4-{4-[(furan-2- ylmethyl)-carbamoyl]-phenyl}-7- isopropyl-5-oxo-6,7-dihydro-5H- pyrrolo[3,4-b]pyridine-3-carboxylic acid butyl ester | 586 |
| 20 | 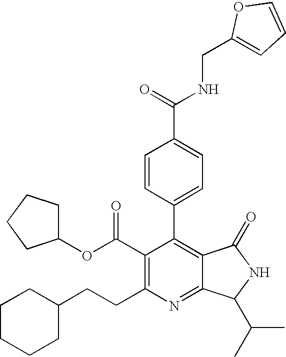 | 2-(2-Cyclohexyl-ethyl)-4-{4-[(furan-2- ylmethyl)-carbamoyl]-phenyl}-7- isopropyl-5-oxo-6,7-dihydro-5H- pyrrolo[3,4-b]pyridine-3-carboxylic acid cyclopentyl ester | 598 |
| 21 | 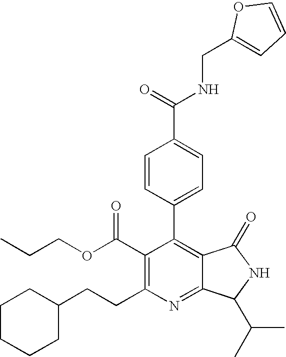 | 2-(2-Cyclohexyl-ethyl)-4-{4-[(furan-2- ylmethyl)-carbamoyl]-phenyl}-7- isopropyl-5-oxo-6,7-dihydro-5H- pyrrolo[3,4-b]pyridine-3-carboxylic acid propyl ester | 572 |
| 22 | 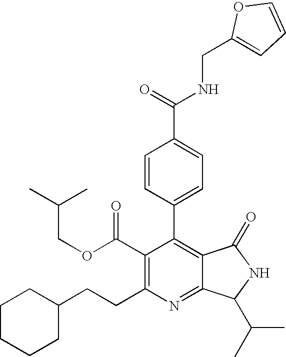 | 2-(2-Cyclohexyl-ethyl)-4-{4-[(furan-2- ylmethyl)-carbamoyl]-phenyl}-7- isopropyl-5-oxo-6,7-dihydro-5H- pyrrolo[3,4-b]pyridine-3-carboxylic acid isobutyl ester | 586 |
| 23 | 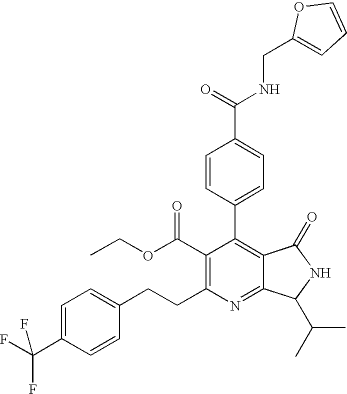 | 4-{4-[(Furan-2-ylmethyl)-carbamoyl]- phenyl}-7-isopropyl-5-oxo-2-[2-(4- trifluoromethyl-phenyl)-ethyl]-6,7- dihydro-5H-pyrrolo[3,4-b]pyridine-3- carboxylic acid ethyl ester | 620 |
| 24 | 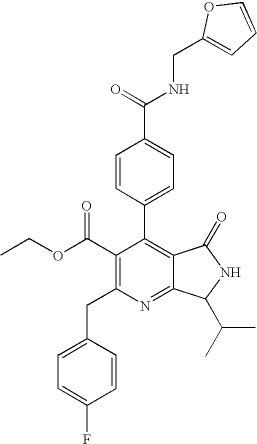 | 2-(4-Fluoro-benzyl)-4-{4-[(furan-2- ylmethyl)-carbamoyl]-phenyl}-7- isopropyl-5-oxo-6,7-dihydro-5H- pyrrolo[3,4-b]pyridine-3-carboxylic acid ethyl ester | 556 |
| 25 | 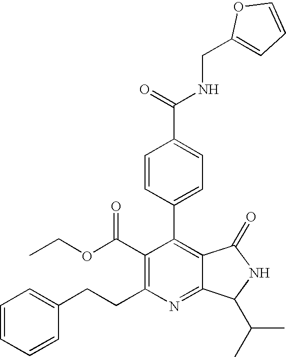 | 4-{4-[(Furan-2-ylmethyl)-carbamoyl]- phenyl}-7-isopropyl-5-oxo-2-phenethyl- 6,7-dihydro-5H-pyrrolo[3,4-b]pyridine-3- carboxylic acid ethyl ester | 552 |
| 26 | 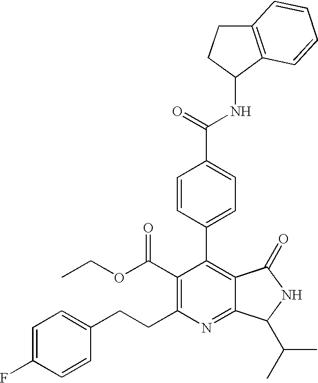 | 2-[2-(4-Fluoro-phenyl)-ethyl]-4-[4-(indan- 1-ylcarbamoyl)-phenyl]-7-isopropyl-5- oxo-6,7-dihydro-5H-pyrrolo[3,4- b]pyridine-3-carboxylic acid ethyl ester | 606 |
| 27 | 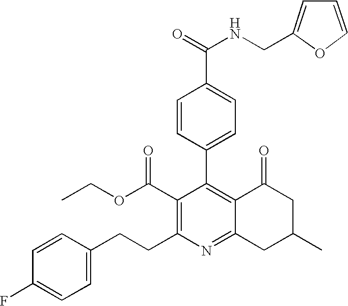 | 2-[2-(4-Fluoro-phenyl)-ethyl]-4-{4- [(furan-2-ylmethyl)-carbamoyl]-phenyl}- 7-methyl-5-oxo-5,6,7,8-tetrahydro- quinoline-3-carboxylic acid ethyl ester | 555 |
| 28 | 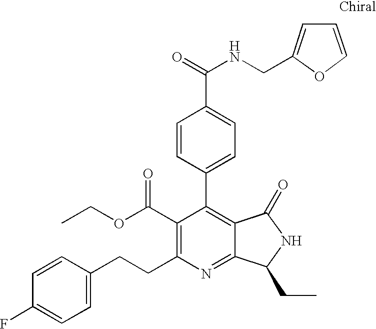 | (S)-7-Ethyl-2-[2-(4-fluoro-phenyl)-ethyl]- 4-{4-[(furan-2-ylmethyl)-carbamoyl]- phenyl}-5-oxo-6,7-dihydro-5H- pyrrolo[3,4-b]pyridine-3-carboxylic acid ethyl ester | 556 |
| 29 | 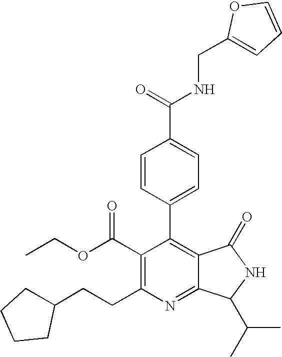 | 2-(2-Cyclopentyl-ethyl)-4-{4-[(furan-2- ylmethyl)carbamoyl]-phenyl}-7- isopropyl-5-oxo-6,7-dihydro-5H- pyrrolo[3,4-b]pyridine-3-carboxylic acid ethyl ester | 544 |
| 30 | 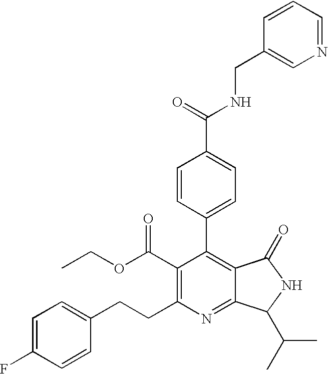 | 2-[2-(4-Fluoro-phenyl)-ethyl]-7-isopropyl- 5-oxo-4-{4-[(pyridin-3-ylmethyl)- carbamoyl]-phenyl}-6,7-dihydro-5H- pyrrolo[3,4-b]pyridine-3-carboxylic acid ethyl ester | 581 |
| 31 | 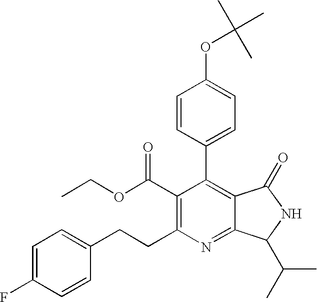 | 4-(4-tert-Butoxy-phenyl)-2-[2-(4-fluoro- phenyl)-ethyl]-7-isopropyl-5-oxo-6,7- dihydro-5H-pyrrolo[3,4-b]pyridine-3- carboxylic acid ethyl ester | 519 |
| 32 | 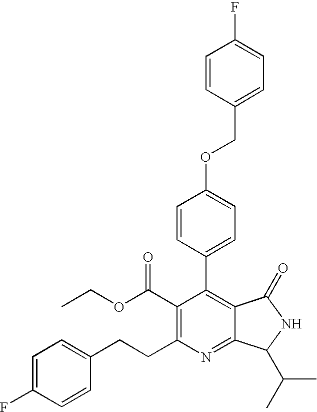 | 4-[4-(4-Fluoro-benzyloxy)-phenyl]-2-[2- (4-fluoro-phenyl)-ethyl]-7-isopropyl-5- oxo-6,7-dihydro-5H-pyrrolo[3,4- b]pyridine-3-carboxylic acid ethyl ester | 571 |
| 33 | 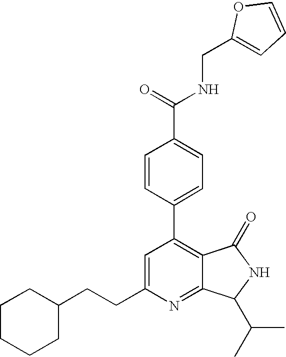 | 4-[2-(2-Cyclohexyl-ethyl)-7-isopropyl-5- oxo-6,7-dihydro-5H-pyrrolo[3,4- b]pyridin-4-yl]-N-furan-2-ylmethyl- benzamide | 486 |
| 34 | 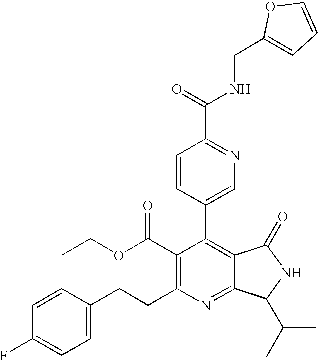 | (S)-2-[2-(4-Fluoro-phenyl)-ethyl]-4-{6- [(furan-2-ylmethyl)-carbamoyl]-pyridin-3- yl}-7-isopropyl-5-oxo-6,7-dihydro-5H- pyrrolo[3,4-b]pyridine-3-carboxylic acid ethyl ester | 571 |
| 35 | 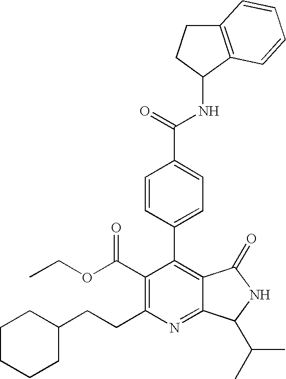 | 2-(2-Cyclohexyl-ethyl)-4-[4-(indan-1- ylcarbamoyl)-phenyl]-7-isopropyl-5-oxo- 6,7-dihydro-5H-pyrrolo[3,4-b]pyridine-3- carboxylic acid ethyl ester | 594 |
| 36 | 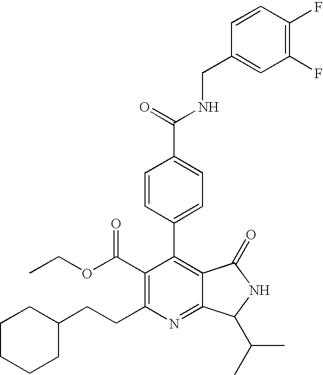 | 2-(2-Cyclohexyl-ethyl)-4-[4-(3,4-difluoro- benzylcarbamoyl)-phenyl]-7-isopropyl-5- oxo-6,7-dihydro-5H-pyrrolo[3,4- b]pyridine-3-carboxylic acid ethyl ester | 604 |
| 37 | 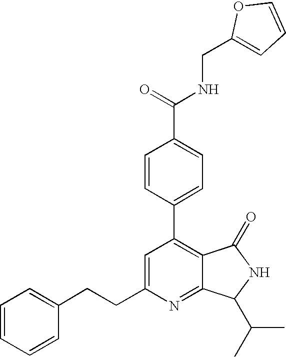 | N-Furan-2-ylmethyl-4-(7-isopropyl-5- oxo-2-phenethyl-6,7-dihydro-5H- pyrrolo[3,4-b]pyridin-4-yl)-benzamide | 480 |
| 38 | 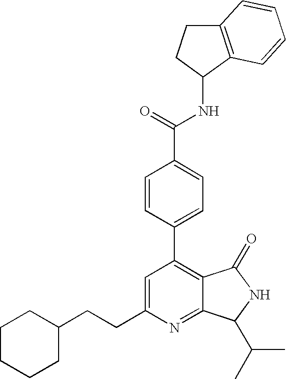 | 4-[(S)-2-(2-Cyclohexyl-ethyl)-7- isopropyl-5-oxo-6,7-dihydro-5H- pyrrolo[3,4-b]pyridin-4-yl]-N-indan-1-yl- benzamide | 522 |

While typical embodiments have been illustrated and described, it should be understood that changes and modifications can be made therein in accordance with ordinary skill in the art without departing from the invention in its broader aspects as defined in the following claims.

1. A compound having formula I, 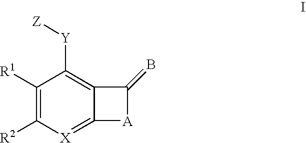
stereoisomers thereof, tautomers thereof, solvates thereof, and pharmaceutically acceptable salts thereof; wherein
A is —NH—CR^{3a}R^{3b}— wherein the NH is attached to the carbon bearing B, or A is —CRR′—CR^{4a}R^{4b}—CRR′—;
B is O or S;
X is N or NO;
Y is a divalent substituted or unsubstituted aryl, heterocyclyl, or cycloalkyl group;
Z is —C(O)OR⁵, —C(O)NR⁶R⁷, —NR⁶C(O)R⁵, —NR⁶C(O)NR⁶R⁷, —C(O)R⁵, —NR⁶R⁷, —OR⁸, —SO₂NR⁶R⁷, —NR⁶SO₂R⁵ or —S(O)ₘR⁵;
R and R′ at each occurrence are independently H or an unsubstituted C₁₋₃ alkyl group;
R¹ is H, —C(O)OR⁹, —C(O)NR¹⁰R¹¹, —CN, —C(O)R⁹ or —NR¹⁰C(O)R⁹;
R² is —(C₁₋₂ alkyl)-R¹² wherein the C₁₋₂ alkyl is substituted or unsubstituted;
R^{3a} is a C₂₋₃ alkyl group, optionally substituted with one or more F;
R^{3b} is H or a C₂₋₃ alkyl group, optionally substituted with one or more F;
R^{4a} and R^{4b} are each independently —H, or substituted or unsubstituted C₁₋₆ alkyl;
R⁵ is a substituted or unsubstituted aralkyl, heteroaralkyl, heterocyclylalkyl, alkyl-cycloalkyl, fused cycloalkylaryl, or fused heterocyclylaryl group;
R⁶ and R⁷ are each independently —H or a substituted or unsubstituted aralkyl, heteroaralkyl, heterocyclylalkyl, alkyl-cycloalkyl, fused cycloalkylaryl, or fused heterocyclylaryl group; or R⁶ and R⁷, when attached to the same atom, together form a substituted or unsubstituted heterocyclyl group;
R⁸ is a substituted or unsubstituted, branched or unbranched C₂₋₆ alkyl, C₂₋₆ alkenyl, C₂₋₆ alkynyl or C₇₋₁₀ aralkyl group;
R⁹ is —H or a substituted or unsubstituted alkyl, heteroalkyl, alkenyl, alkynyl, aryl, aralkyl, heterocyclyl, or heterocyclylalkyl group;
R¹⁰ and R¹¹ are each independently —H or a substituted or unsubstituted alkyl, heteroalkyl, alkenyl, alkynyl, aryl, aralkyl, heterocyclyl, heterocyclylalkyl group; or R¹⁰ and R¹¹, when attached to the same atom, together form a substituted or unsubstituted heterocyclyl group;
R¹² is a substituted or unsubstituted cycloalkyl, aryl or heteroaryl group or an unsubstituted alkyl group; and
m is 0, 1 or 2.

2. The compound of claim 1 wherein A is —NH—CR^{3a}R^{3b}—.

3. The compound of claim 1 wherein B is O.

4. The compound of claim 1 wherein X is N.

5. The compound of claim 1 wherein Y is a divalent substituted or unsubstituted aryl or heterocyclyl group.

6. The compound of claim 1 wherein Y is a divalent aryl or heteroaryl group.

7. The compound of claim 1 wherein Y is a divalent phenyl group.

8. The compound of claim 1 wherein Z is —C(O)OR⁵, —C(O)NR⁶R⁷, —NR⁶C(O)R⁵, —C(O)R⁵, —OR⁸, —SO₂NR⁶R⁷, —NR₆R₇SO₂R₅ or —S(O)ₘR⁵.

9. The compound of claim 1 wherein Z is —C(O)OR⁵, —C(O)NR⁶R⁷, or —OR⁸.

10. The compound of claim 1 wherein R¹ is H, —C(O)OR⁹ or —C(O)NR¹⁰R¹¹.

11. The compound of claim 1 wherein R⁹, R¹⁰ and R¹¹ are each independently —H, methyl, ethyl, n-propyl, n-butyl, isobutyl, sec-butyl, tert-butyl, cyclopentyl, or allyl.

12. The compound of claim 1 wherein R² is —CH₂—CH₂—R¹².

13. The compound of claim 1 wherein R¹² is cyclohexyl, cyclopentyl, phenyl, 4-fluorophenyl, 4-trifluorophenyl, isobutyl or t-butyl.

14. The compound of claim 1 having the Formula II:

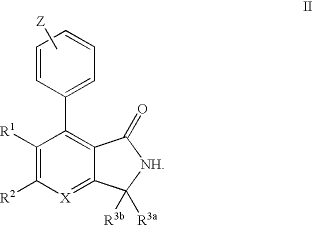

15. The compound of claim 14 wherein Z is —C(O)OR⁵, —C(O)NR⁶R⁷, or —OR⁸.

16. The compound of claim 1 having the Formula III:

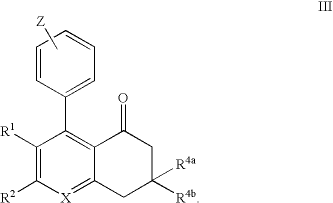

17. The compound of claim 16 wherein Z is —C(O)OR⁵, —C(O)NR⁶R⁷, or —OR⁸.

18. A compound of claim 1 wherein the compound is
2-[2-(4-Fluoro-phenyl)-ethyl]-4-{4-[(furan-2-ylmethyl)-carbamoyl]-phenyl}-7-isopropyl-5-oxo-6,7-dihydro-5H-pyrrolo[3,4-b]pyridine-3-carboxylic acid ethyl ester,
4-{2-[2-(4-Fluoro-phenyl)-ethyl]-7-isopropyl-5-oxo-6,7-dihydro-5H-pyrrolo[3,4-b]pyridin-4-yl}-N-furan-2-ylmethyl-benzamide,
2-[2-(4-Fluoro-phenyl)-ethyl]-4-{4-[(furan-2-ylmethyl)-carbamoyl]-phenyl}-7,7-dimethyl-5-oxo-5,6,7,8-tetrahydro-quinoline-3-carboxylic acid ethyl ester,
2-(2-Cyclohexyl-ethyl)-4-{4-[(furan-2-ylmethyl)-carbamoyl]-phenyl}-7-isopropyl-5-oxo-6,7-dihydro-5H-pyrrolo[3,4-b]pyridine-3-carboxylic acid ethyl ester,
2-(2-Cyclohexyl-ethyl)-4-{4-[(furan-2-ylmethyl)-carbamoyl]-phenyl}-7-isopropyl-5-oxo-6,7-dihydro-5H-pyrrolo[3,4-b]pyridine-3-carboxylic acid methyl ester,
2-(2-Cyclohexyl-ethyl)-4-{4-[(furan-2-ylmethyl)-carbamoyl]-phenyl}-7-isopropyl-5-oxo-6,7-dihydro-5H-pyrrolo[3,4-b]pyridine-3-carboxylic acid allyl ester,
2-(2-Cyclohexyl-ethyl)-4-{4-[(furan-2-ylmethyl)-carbamoyl]-phenyl}-7-isopropyl-5-oxo-6,7-dihydro-5H-pyrrolo[3,4-b]pyridine-3-carboxylic acid isopropyl ester,
2-(2-Cyclohexyl-ethyl)-4-{4-[(furan-2-ylmethyl)-carbamoyl]-phenyl}-7-isopropyl-5-oxo-6,7-dihydro-5H-pyrrolo[3,4-b]pyridine-3-carboxylic acid butyl ester,
2-(2-Cyclohexyl-ethyl)-4-{4-[(furan-2-ylmethyl)-carbamoyl]-phenyl}-7-isopropyl-5-oxo-6,7-dihydro-5H-pyrrolo[3,4-b]pyridine-3-carboxylic acid cyclopentyl ester,
2-(2-Cyclohexyl-ethyl)-4-{4-[(furan-2-ylmethyl)-carbamoyl]-phenyl}-7-isopropyl-5-oxo-6,7-dihydro-5H-pyrrolo[3,4-b]pyridine-3-carboxylic acid propyl ester,
2-(2-Cyclohexyl-ethyl)-4-{4-[(furan-2-ylmethyl)-carbamoyl]-phenyl}-7-isopropyl-5-oxo-6,7-dihydro-5H-pyrrolo[3,4-b]pyridine-3-carboxylic acid isobutyl ester,
4-{4-[(Furan-2-ylmethyl)-carbamoyl]-phenyl}-7-isopropyl-5-oxo-2-[2-(4-trifluoromethyl-phenyl)-ethyl]-6,7-dihydro-5H-pyrrolo[3,4-b]pyridine-3-carboxylic acid ethyl ester,
2-(4-Fluoro-benzyl)-4-{4-[(furan-2-ylmethyl)-carbamoyl]-phenyl}-7-isopropyl-5-oxo-6,7-dihydro-5H-pyrrolo[3,4-b]pyridine-3-carboxylic acid ethyl ester,
4-{4-[(Furan-2-ylmethyl)-carbamoyl]-phenyl}-7-isopropyl-5-oxo-2-phenethyl-6,7-dihydro-5H-pyrrolo[3,4-b]pyridine-3-carboxylic acid ethyl ester,
2-[2-(4-Fluoro-phenyl)-ethyl]-4-[4-(indan-1-ylcarbamoyl)-phenyl]-7-isopropyl-5-oxo-6,7-dihydro-5H-pyrrolo[3,4-b]pyridine-3-carboxylic acid ethyl ester,
2-[2-(4-Fluoro-phenyl)-ethyl]-4-{4-[(furan-2-ylmethyl)-carbamoyl]-phenyl}-7-methyl-5-oxo-5,6,7,8-tetrahydro-quinoline-3-carboxylic acid ethyl ester,
(S)-7-Ethyl-2-[2-(4-fluoro-phenyl)-ethyl]-4-{4-[(furan-2-ylmethyl)-carbamoyl]-phenyl}-5-oxo-6,7-dihydro-5H-pyrrolo[3,4-b]pyridine-3-carboxylic acid ethyl ester,
2-(2-Cyclopentyl-ethyl)-4-{4-[(furan-2-ylmethyl)-carbamoyl]-phenyl}-7-isopropyl-5-oxo-6,7-dihydro-5H-pyrrolo[3,4-b]pyridine-3-carboxylic acid ethyl ester,
2-[2-(4-Fluoro-phenyl)-ethyl]-7-isopropyl-5-oxo-4-{4-[(pyridin-3-ylmethyl)-carbamoyl]-phenyl}-6,7-dihydro-5H-pyrrolo[3,4-b]pyridine-3-carboxylic acid ethyl ester,
4-(4-tert-Butoxy-phenyl)-2-[2-(4-fluoro-phenyl)-ethyl]-7-isopropyl-5-oxo-6,7-dihydro-5H-pyrrolo[3,4-b]pyridine-3-carboxylic acid ethyl ester
4-[4-(4-Fluoro-benzyloxy)-phenyl]-2-[2-(4-fluoro-phenyl)-ethyl]-7-isopropyl-5-oxo-6,7-dihydro-5H-pyrrolo[3,4-b]pyridine-3-carboxylic acid ethyl ester,
4-[2-(2-Cyclohexyl-ethyl)-7-isopropyl-5-oxo-6,7-dihydro-5H-pyrrolo[3,4-b]pyridin-4-yl]-N-furan-2-ylmethyl-benzamide,
(S)-2-[2-(4-Fluoro-phenyl)-ethyl]-4-{6-[(furan-2-ylmethyl)-carbamoyl]-pyridin-3-yl}-7-isopropyl-5-oxo-6,7-dihydro-5H-pyrrolo[3,4-b]pyridine-3-carboxylic acid ethyl ester,
2-(2-Cyclohexyl-ethyl)-4-[4-(indan-1-ylcarbamoyl)-phenyl]-7-isopropyl-5-oxo-6,7-dihydro-5H-pyrrolo[3,4-b]pyridine-3-carboxylic acid ethyl ester,
2-(2-Cyclohexyl-ethyl)-4-[4-(3,4-difluoro-benzylcarbamoyl)-phenyl]-7-isopropyl-5-oxo-6,7-dihydro-5H-pyrrolo[3,4-b]pyridine-3-carboxylic acid ethyl ester,
N-Furan-2-ylmethyl-4-(7-isopropyl-5-oxo-2-phenethyl-6,7-dihydro-5H-pyrrolo[3,4-b]pyridin-4-yl)-benzamide, or
4-[(S)-2-(2-Cyclohexyl-ethyl)-7-isopropyl-5-oxo-6,7-dihydro-5H-pyrrolo[3,4-b]pyridin-4-yl]-N-indan-1-yl-benzamide.

19. A method of preparing a compound of claim 1 wherein A is —NH—CR^{3a}R^{3b}— and X is N, the method comprising exposing a compound of formula IV 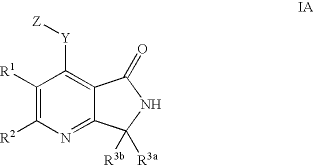
to a first oxidant that selectively oxidizes a dihydropyridinyl ring to a pyridinyl ring, thereby providing a compound of formula IA 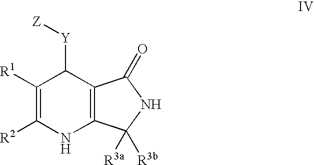
wherein Z, Y, R¹, R², R^{3a}, and R^{3b} are as defined previously.

20-32. (canceled)

33. A composition comprising a compound of claim 1 and a pharmaceutically acceptable carrier.

34. A method of treating a disorder mediated by a calcitonin or amylin receptor, the method comprising administering to a subject in need thereof a therapeutically effective amount of a compound of claim 1.

35. The method of claim 34 wherein the compound is a calcitonin or amylin receptor agonist.

36. The method of claim 34 wherein the disorder is osteoporosis, Paget's disease, hypercalcemia, Sudeck's atrophy, polystatic fibrous displasia, intersemocostoclavicular ossification, osteogenesis imperfecta, osteopenia, periodontal disease or defect, osteolytic bone disease, metastatic bone disorder, bone loss resulting from malignancy, endocrine disorder, autoimmune arthritides, breakage and fracture, or immobility and disuse, osteopathic pain, phantom limb pain, general pain, hyperalgesia, pain associated with diabetic neuropathy, insulin dependent diabetes, non-insulin dependent diabetes, impaired glucose tolerance, obesity, syndrome X, diabetic complications, primary or secondary hyperthyroidism, conditions associated with inhibiting gastric secretion, gastrointestinal disorders, renal osteodystrophy, obesity, or male infertility.

37. A method of treating a bone disorder, the method comprising administering to a subject in need thereof a therapeutically effective amount of a compound of claim 1.

38. The method of claim 37 wherein the bone disorder is osteoporosis, Paget's disease, hypercalcemia, Sudeck's atrophy, polystatic fibrous displasia, intersemocostoclavicular ossification, osteogenesis imperfecta, osteopenia, periodontal disease or defect, osteolytic bone disease, metastatic bone disorder, bone loss resulting from malignancy, autoimmune arthritides, breakage and fracture, or immobility and disuse.

39. The method of claim 37 wherein the bone disorder is osteoporosis or Paget's disease.

40. A method of treating pain, the method comprising administering to a subject in need thereof a therapeutically effective amount of a compound of claim 1.

41. The method of claim 40 wherein the pain is osteopathic pain, phantom limb pain, general pain, hyperalgesia, or pain associated with diabetic neuropathy.

42. A method of treating a metabolic disorder, the method comprising administering to a subject in need thereof a therapeutically effective amount of a compound of claim 1.

43. The method of claim 42 wherein the metabolic disorder is insulin dependent diabetes, non-insulin dependent diabetes, impaired glucose tolerance, obesity, syndrome X, diabetes mellitus, hypoglycemia or an insulin-requiring disorder other than diabetes mellitus or diabetic complications.

44. A method of modulating the activity of a calcitonin or amylin receptor comprising contacting the calcitonin or amylin receptor with a compound of claim 1.

45. The method of claim 44 wherein the compound is an agonist at the calcitonin or amylin receptor.

46. The method of claim 43 wherein the compound is administered in combination with an insulin for the treatment of diabetes mellitus or an insulin-requiring disorder other than diabetes mellitus.

47. The method of claim 43 wherein the compound is administered in combination with a glucagon for the treatment of hypoglycemia.
